(19) United States
(12) Patent Application Publication
(10) Pub. No.: US 2022/0002661 A1
(43) Pub. Date: Jan. 6, 2022

(54) MODULATION OF FORMATE OXIDATION BY RECOMBINANT YEAST HOST CELL DURING FERMENTATION

(71) Applicant: Lallemand Hungary Liquidity Management LLC, Budapest (HU)

(72) Inventors: Trisha Barrett, Bradford, VT (US); Ryan Skinner, Bethel, VT (US); Aaron Argyros, Lebanon, NH (US)

(21) Appl. No.: 17/292,991

(22) PCT Filed: Nov. 13, 2019
(86) PCT No.: PCT/IB2019/059760

§ 371 Date: May 11, 2021

Related U.S. Application Data
(60) Provisional application No. 62/760,444, filed on Nov. 13, 2018.

(51) Int. Cl. C12N 1/16; C12N 9/02; C12N 9/10

(52) U.S. Cl. CPC C12N 1/16; C12N 9/0093; C12N 9/1029; C12R 2001/865; C12Y 203/01054; C12Y 117/01; C12Y 197/01004

(57) ABSTRACT

The present disclosure concerns recombinant yeast host cells having a first genetic modification for increasing formate production, when compared to a corresponding native yeast host cell as well as a source of formate dehydrogenase activity. The source of formate can be an internal source of formate dehydrogenase activity and/or the recombinant yeast host call can be supplemented by an external source of formate dehydrogenase activity.

### CROSS-REFERENCE TO RELATED APPLICATIONS AND SEQUENCE LISTING STATEMENT

This application claims priority from U.S. provisional application Ser. No. 62/760,444 filed on Nov. 13, 2018 and herewith incorporated in its entirety. The sequence listing associated with this application is provided in text format in lieu of a paper copy and is hereby incorporated by reference into the specification. The name of the text file containing the sequence listing is PCT_-_Sequence_listing_as_filed. The text file is 90 Ko, was created on Nov. 13, 2019 and is being submitted electronically.

### TECHNOLOGICAL FIELD

The present disclosure concerns a recombinant yeast host cell oxidizing formate during fermentation.

### BACKGROUND

Saccharomyces cerevisiae is the primary biocatalyst used in the commercial production of fuel ethanol. This organism is proficient in fermenting glucose to ethanol, often to concentrations greater than 20% (v/v). To further improve upon this ethanol yield, utilization of formate production as an alternate to glycerol as an electron sink, which results in reduced glycerol production, has been engineered into yeast (e.g., WO2012138942). This strategy successfully reduces the secretion of the fermentation by-product glycerol, and increases valuable ethanol production by the strain.

It would be highly desirable to be provided with alternative recombinant yeast host cell which would provide increased yield during fermentation, especially fermentation conducted in the presence of a stressor.

### BRIEF SUMMARY

The present disclosures provides for recombinant yeast host cell having an increase level of formate a source of formate dehydrogenase activity. This source of formate dehydrogenase activity can be especially useful during fermentation for increasing or maintaining the fermentation yield (especially in the presence of a stressor), limiting glycerol production and/or increasing glucose uptake.

According to a first aspect, the present disclosure provides a recombinant yeast host cell having (i) a first genetic modification for increasing formate production, when compared to a corresponding native yeast host cell and (ii) a source of formate dehydrogenase activity. The source of formate dehydrogenase activity can be an internal source of formate dehydrogenase activity provided by a second genetic modification. Alternatively or in combination, the source of formate dehydrogenase activity can be an external source of formate dehydrogenase activity provided by a further yeast host cell having a third genetic modification. In an embodiment, the first genetic modification comprises introducing one or more first heterologous nucleic acid molecule encoding one or more polypeptide having pyruvate formate lyase activity in the recombinant yeast host cell. In a specific embodiment, the one or more polypeptide having pyruvate formate lyase activity comprises PFLA, PFLB or a combination thereof. In another specific embodiment, the one or more polypeptide having pyruvate formate lyase activity comprises PFLA and PFLB. In an embodiment, the one or more polypeptide having pyruvate formate lyase activity is from Bifidobacterium. In still another embodiment, the one or more polypeptide having pyruvate formate lyase activity is from Bifidobacterium adolescentis. In still another embodiment, the one or more polypeptide having pyruvate formate lyase activity comprises the amino acid sequence of SEQ ID NO: 6, is a variant of the amino acid sequence of SEQ ID NO: 6 having pyruvate formate lyase activity or is a fragment of the amino acid sequence of SEQ ID NO: 6 having pyruvate formate lyase activity. In still a further embodiment, the one or more polypeptide having pyruvate formate lyase activity comprises the amino acid sequence of SEQ ID NO: 7, is a variant of the amino acid sequence of SEQ ID NO: 7 having pyruvate formate lyase activity or is a fragment of the amino acid sequence of SEQ ID NO: 7 having pyruvate formate lyase activity. In an embodiment, the second and/or third genetic modification comprises introducing a second and/or third heterologous nucleic acid molecule encoding a polypeptide having formate dehydrogenase activity. In an embodiment, the polypeptide having formate dehydrogenase activity is FDH1. In still another embodiment, the polypeptide having formate dehydrogenase activity uses NAD⁺ as a primary cofactor. For example, the polypeptide having formate dehydrogenase activity (and using NAD⁺ as a primary cofactor) can have the amino acid sequence of SEQ ID NO: 1 or 5, be a variant of the amino acid sequence of SEQ ID NO: 1 or 5 having formate dehydrogenase activity or be a fragment of the amino acid sequence of SEQ ID NO: 1 or 5 having formate dehydrogenase activity. In another embodiment, the polypeptide having formate dehydrogenase activity uses NADP⁺ as a primary cofactor. For example, the polypeptide having formate dehydrogenase activity (and using NADP⁺ as a primary cofactor) can have the amino acid sequence of SEQ ID NO: 2, 3, 4, 21, 23, 25, 26 or 27, be a variant of the amino acid sequence of SEQ ID NO: 2, 3, 4, 21, 23, 25, 26 or 27 having formate dehydrogenase activity or be a fragment of the amino acid sequence of SEQ ID NO: 2, 3, 4, 21, 23, 25, 26 or 27 having formate dehydrogenase activity. In yet another embodiment, the second and/or third heterologous nucleic acid molecule has a mitochondrial target sequence operatively associated with the nucleic acid sequence encoding the polypeptide having formate dehydrogenase activity. In a specific embodiment, the mitochondrial target sequence is from the CYB2 gene and can have, for example, the amino acid sequence of SEQ ID NO: 11, is a variant of the amino acid sequence of SEQ ID NO: 11 or is a fragment of the amino acid sequence of SEQ ID NO: 11. 17. In another embodiment, the second and/or third heterologous nucleic acid molecule further comprises a promoter operatively associated with the nucleic acid sequence encoding the polypeptide having formate dehydrogenase activity. In some embodiments, the promoter can comprise at least one of tef2p, ssa1p, adh1p, cdc19p, tpi1p, cyc1p, pgk1p, tdh2p, eno2p, hxt3p, qcr8p, tdh1p, tdh3p or hor7p as well as combinations thereof. In an embodiment, the recombinant yeast host cell expresses native FDH gene(s). In another embodiment, the further yeast host cell expresses native FDH gene(s). In still another embodiment, the recombinant yeast host cell comprises a fourth genetic modification for invactivating of at least one of the native FDH gene(s). In still another embodiment, the further yeast host cell comprises a fifth genetic modification invactivating of at least one of the native FDH gene(s). In a further embodiment, the native FDH gene(s) comprises FDH1, FDH2 or both. In an embodiment, the recombinant yeast host cell is from the genus Saccharomyces, for example from the species Saccharomyces cerevisiae. In another embodiment, the further yeast host cell is from the genus Saccharomyces, for example from the species Saccharomyces cerevisiae.

According to a second aspect, the present disclosure provides a combination for fermenting a biomass, the combination comprising the recombinant yeast host cell defined in herein and the further yeast host cell defined herein. In an embodiment, at least one or both of the recombinant yeast host cell or the further yeast host cell is provided as a cream.

According to a third aspect, the present disclosure provides a process for converting a biomass into a fermentation product, the process comprises contacting the biomass with the recombinant yeast host cell defined herein, optionally in combination with the further yeast host cell defined herein, or the combination defined herein under condition to allow the conversion of at least a part of the biomass into the fermentation product. In an embodiment, the biomass comprises corn which can optionally provided as a mash. In yet another embodiment, the fermentation product is ethanol. In some embodiment, the process is being conducted, at least in part, in the presence of a stressor such as, for example, lactic acid, formic acid and/or a bacterial contamination.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the impact of modulating FDH1 copy number on ethanol and glycerol production during a permissive fermentation. Results are shown as ethanol (g/L, left axis bars) and glycerol (g/L, right axis, ▴) content after 50 h of permissive fermentation for strains M2390, M8841, M12156, M15052, M15418 and M15419. The formate content obtained after the permissive fermentation is shown in Table 1 below.

TABLE 1
Formate content (g/L) after 50 h of permissive fermentation.

| M2390 | M8841 | M12156 | M15052 | M15418 | M15419 |
|---|---|---|---|---|---|
| 0.050 | 0.190 | 0.450 | 0.000 | 0.190 | 0.090 |

FIG. 2 illustrates the impact of modulating FDH1 copy number on ethanol production and glucose consumption during a lactic stress fermentation. Results are shown as ethanol (g/L, left axis, bars) and glucose (g/L, right axis, ▴) content after 50 h of lactic stress fermentation for strains M2390, M8841, M12156, M15052, M15418 and M15419. The formate content obtained after the lactic stress fermentation is shown in Table 2 below.

TABLE 2
Formate content (g/L) after 50 h of lactic stress fermentation.

| M2390 | M8841 | M12156 | M15052 | M15418 | M15419 |
|---|---|---|---|---|---|
| 0.050 | 0.135 | 0.350 | 0.000 | 0.130 | 0.050 |

FIG. 3 illustrates the impact of expressing an heterologous formate dehydrogenase as well as targeting the expression of a formate dehydrogenase to the mitochondria on ethanol and glycerol production during a permissive fermentation. Results are shown as ethanol (g/L, left axis, bars) and glycerol (g/L, right axis, ▴) content after 50 h of permissive fermentation for strains M2390, M8841, M12156, M15052, M15425, M15427 and M15430. The formate content obtained after the permissive fermentation is shown in Table 3 below.

TABLE 3
Formate content (g/L) after 50 h of permissive fermentation.

| M2390 | M8841 | M12156 | M15052 | M15425 | M15427 | M15430 |
|---|---|---|---|---|---|---|
| 0.050 | 0.190 | 0.450 | 0.000 | 0.000 | 0.290 | 0.070 |

FIG. 4 illustrates the impact of expressing an heterologous formate dehydrogenase as well as targeting the expression of a formate dehydrogenase to the mitochondria on ethanol production and glucose consumption during a lactic stress fermentation. Results are shown as ethanol (g/L, left axis, bars) and glucose (g/L, right axis, ▴) content after 50 h of lactic stress fermentation for strains M2390, M8841, M12156, M15052, M15425, M15427 and M15430. The formate content obtained after the lactic stress fermentation is shown in Table 4 below.

TABLE 4
Formate content (g/L) after 50 h of lactic stress fermentation.

| M2390 | M8841 | M12156 | M15052 | M15425 | Ml5427 | M15430 |
|---|---|---|---|---|---|---|
| 0.050 | 0.135 | 0.350 | 0.000 | 0.000 | 0.280 | 0.070 |

FIGS. 5A and B illustrate the impact of expressing an heterologous formate dehydrogenase as well as targeting the expression of a formate dehydrogenase to the mitochondria on ethanol and glycerol production as well as glucose consumption during (FIG. 5A) a permissive fermentation and (FIG. 5B) a lactic stress fermentation. Results are shown as ethanol (g/L, left axis on both FIGS. 5A and 5B, bars), glycerol (g/L, right axis on FIG. 5A only, ▴) and glucose (g/L, right axis on FIG. 5B only, ▴) content after 50 h of fermentation for strains M2390, M8841, M12156, M15419 and M15430. The formate content obtained after the fermentations is shown in Table 5 below.

TABLE 5
Formate content (g/L) after 50 h of fermentation.

| | M2390 | M8841 | M12156 | M15419 | M15430 |
|---|---|---|---|---|---|
| 5A -Permissive | 0.03 | 0.06 | 0.14 | 0.04 | 0.05 |
| 5B - Lactic | 0.08 | 0.12 | 0.22 | 0.08 | 0.10 |

FIG. 6 illustrates the effects of formate dehydrogenase expression in permissive or stressful fermentations. Results are shown as ethanol (g/L, bars, left axis) and glycerol (g/L, right axis, ▴) content during permissive, lactic/formic or bacterial/formic fermentations for strains M2390, M8841, M12156, M15419 and M15430. The formate content obtained after fermentation is shown in Table 6.

TABLE 6
Format content (units) after 50 h of fermentation.

| M2390 | | | M8841 | | | M12156 | | | M15419 | | | M15430 | | |
|---|---|---|---|---|---|---|---|---|---|---|---|---|---|---|
| P | LF | BF | P | LF | BF | P | LF | BF | P | LF | BF | P | LF | BF |
| 0.000 | 0.035 | 0.070 | 0.000 | 0.060 | 0.100 | 0.000 | 0.095 | 0.340 | 0.000 | 0.000 | 0.000 | 0.000 | 0.010 | 0.015 |

P = permissive, LF = lactic and formic stress fermentation, BF = bacterial and formic stress fermentation.

FIGS. 7A and 7B illustrate the impact of blending a strain overexpressing a formate dehydrogenase with a strain which does not express a formate dehydrogenase during permissive and lactic stress fermentations. (FIG. 7A) Results are shown as ethanol content (g/L) during permissive (standard) and lactic stress fermentations for strains M2390, M8841, M12156, M15419 alone or in combination with M12156 (either 50/50 or 90 (M12156)/10 (M15419)). (FIG. 7B) Additional results are shown as ethanol content (g/L) during permissive (standard) and lactic stress fermentations for strains M2390, M8841, M12156, M15430 alone or in combination with M12156 (either 50/50 or 90(M12156)/10(M15430)) during permissive or lactic stress fermentation. The formate content obtained after the fermentations is shown in Table 7 below.

TABLE 7
Formate content (g/L) after 50 h of fermentation.

| | M2390 | | M8841 | | M12156 | | M15419 | | 50/50 | | 90/10 | |
|---|---|---|---|---|---|---|---|---|---|---|---|---|
| | P | L | P | L | P | L | P | L | P | L | P | L |
| A | 0.030 | 0.020 | 0.235 | 0.140 | 0.320 | 0.190 | 0.095 | 0.020 | 0.165 | 0.040 | 0.265 | 0.125 |
| | M2390 | | M8841 | | M12156 | | M15430 | | 50/50 | | 90/10 | |
| | P | L | P | L | P | L | P | L | P | L | P | LL |
| B | 0.030 | 0.020 | 0.235 | 0.140 | 0.320 | 0.190 | 0.090 | 0.025 | 0.125 | 0.050 | 0.270 | 0.155 |

P = permissive fermentation, L = lactic fermentation.

FIG. 8 illustrates the effect of deleting or keeping the endogenous FDH genes on ethanol, glycerol and formate production as well as glucose consumption during permissive and lactic stress fermentations. Results are shown as ethanol (g/L, left axis, bars), glucose (g/L, right axis, ●), glycerol (g/L, right axis, ▪) or formate (g/L, right axis, ♦) content after 48 h of permissive or stress (lactic acid) fermentation for strains M2390, M12156, M15419 and M17952. The formate content obtained after the fermentations is shown in Table 8 below.

TABLE 8
Formate content (units) after 50 h of fermentation.

| | | | |
|---|---|---|---|
| M2390 | M12156 | M15419 | M17952 |

| P | L | P | L | P | L | P | L |
|---|---|---|---|---|---|---|---|
| 0.0 | 0.0 | 0.2 | 0.2 | 0.0 | 0.0 | 0.0 | 0.0 |

P = permissive fermentation, L = lactic fermentation.

FIG. 9 illustrates the NAD+ and NADP+ activity of recombinant yeast host cell expressing the MP1180 (e.g., Lactobacillus buchneri NADP+-dependent FDH) expressed under the control of the adh1 promoter (M20345), tef2 promoter (M220341) or the ssa1 promoter (M20344). Results are shown as the absorbance (nm od NADH or NADPH/min/mg of protein) in function of the strain tested.

FIG. 10 illustrates the impact of expressing both native and heterologous formate dehydrogenases on ethanol production, glucose consumption, glycerol product and formate consumption during a permissive fermentation. Results are shown as ethanol (g/L, left axis, bars), glucose (g/L, left axis, ▪), glycerol (g/L, left axis, ▴) or formate (g/L, right axis, ♦) content after 48 h of permissive stress fermentation for strains M8279, M18971, M20341, M20345, M20344, M20999, M21000 and M21001.

FIG. 11 illustrates the impact of expressing both native and heterologous formate dehydrogenases on ethanol production, glucose consumption, glycerol product and formate consumption during a stress (lactic acid) fermentation. Results are shown as ethanol (g/L, left axis, bars) glucose (g/L, left axis, ▪), glycerol (g/L, left axis, ▴) or formate (g/L, right axis, ♦) content after 65 h of permissive stress fermentation for strains M8279, M18971, M20341, M20345, M20344, M20999, M21000 and M21001.

FIG. 12 illustrates the NAD+ and NADP+ activity of recombinant yeast host cell expressing the MP1180 (e.g., Lactobacillus buchneri NADP+-dependent FDH) expressed under the control of different promoters (see Table 9B for a description of the strains tested) or G199A (SEQ ID NO: 25) or Q222A (SEQ ID NO: 26) FDH expressed under the control of the tef2 promoter. Results are shown as the absorbance (nm of NADH (dark grey bars) or NADPH (light grey bars/min/mg of protein) in function of the strain tested.

### DETAILED DESCRIPTION

While the use of formate as an alternative electron sink has been proven useful to maintain or increase ethanol yield, this strategy can result, as shown in the Examples below, in the accumulation of formate (internally and/or externally in the fermentation medium) during bioprocesses in instances when the fermenting strain lacks the ability to oxidize formate to carbon dioxide via formate dehydrogenase (FDH). This can result in the accumulation of formic acid to toxic levels, thereby limiting the organism's ability from finishing fermentation effectively and/or reducing its robustness in the presence of a stressor. In some instances, the presence of a native FDH gene(s) and activity may not be sufficient to reduce formic acid content to an acceptable level. In addition, numerous mixed acid fermenting bacteria are known to produce formate which can also accumulate and impact all yeast strains. As shown specifically in the Examples below, strains having a reduced or no ability to oxidize formate to carbon dioxide using formate dehydrogenases exhibit reduced robustness especially in fermentations conducted in the presence of a stressor (such as lactic acid, formic acid and/or the presence of bacteria).

The present disclosure thus provides a recombinant yeast host cell which does increase formate production and also exhibits formate dehydrogenase activity so as to maintain or increase the fermentation yield. In an embodiment, when a biomass (for example comprising corn) is fermented by the recombinant yeast host cell of the present disclosure (or the combination comprising the recombinant yeast host cell of the present disclosure), at the conclusion of a fermentation, the fermentation medium has less than 2 g/L, 1.9 g/L, 1.8 g/L, 1.7 g/L, 1.6 g/L, 1.5 g/L, 1.4 g/L, 1.3 g/L, 1.2 g/IL, 1.1 g/L, 1 g/L, 0.9 g/L, 0.8 g/L, 0.7 g/L, 0.6 g/L, 0.5 g/L, 0.4 g/L, 0.3 g/L, 0.2 g/L or 0.1 g/L of formate. Alternatively or in combination, in an embodiment, when a biomass (for example comprising corn) is fermented by the recombinant yeast host cell of the present disclosure (or the combination comprising the recombinant yeast host cell of the present disclosure), at the conclusion of a fermentation, the fermentation medium has less than 12 g/L, 11 g/L, 10 g/L, 9 g/L, 8 g/L, 7 g/L, 6 g/L, 5 g/L, 4 g/L, 3 g/L, 2 g/L or 1 g/L of glycerol. Alternatively or in combination, when a biomass (for example comprising corn) is fermented by the recombinant yeast host cell of the present disclosure (or the combination comprising the recombinant yeast host cell of the present disclosure), at the conclusion of a fermentation, the fermentation medium has less than 10 g/L, 9 g/L, 8 g/L, 7 g/L, 6 g/L, 5 g/L, 4 g/L, 3 g/L, 2 g/L, 1 g/L or less of glucose. Alternatively or in combination, when a biomass (for example comprising corn) is fermented by the recombinant yeast host cell of the present disclosure (or the combination comprising the recombinant yeast host cell of the present disclosure), at the conclusion of a permissive fermentation, the fermentation medium has at least 100 g/L, 105 g/L, 110 g/L, 115 g/L, 120 g/L, 125 g/L, 130 g/L, 135 g/L or 140 g/L of ethanol. Alternatively or in combination, when a biomass (for example comprising corn) is fermented by the recombinant yeast host cell of the present disclosure, at the conclusion of a stress fermentation, the fermentation medium has at least 50 g/L, 55 g/L, 60 g/L, 65 g/L, 70 g/L, 75 g/L, 80 g/L, 85 g/L or 90 g/L of ethanol.

Recombinant Yeast Host Cell

The present disclosure concerns recombinant yeast host cells (which can be provided, in some embodiments, in combination with further yeast host cells). The recombinant yeast host cell are obtained by introducing at least two genetic modifications in a corresponding native yeast host cell and optionally in a further yeast host cell. The genetic modification(s) in the recombinant yeast host cell of the present disclosure comprise, consist essentially of or consist of a first genetic modification for increasing formate production and at least one of a second genetic modification (in the recombinant yeast host cell) or a third genetic modification (in the further yeast host cell) for increasing formate dehydrogenase activity. In the context of the present disclosure, the expression “the genetic modification(s) in the recombinant yeast host consists essentially of a first genetic modification, and at least one of a second genetic modification or a third genetic modification” refers to the fact that the recombinant yeast host cell and further yeast host cell can include other genetic modifications which are unrelated to the anabolism or the catabolism of formate.

When the genetic modification is aimed at reducing or inhibiting the expression of a specific targeted gene (which is endogenous to the host cell), the genetic modifications can be made in one, two or all copies of the targeted gene(s). When the genetic modification is aimed at increasing the expression of a specific targeted gene, the genetic modification can be made in one or multiple genetic locations. In the context of the present disclosure, when recombinant yeast host cells are qualified as being “genetically engineered”, it is understood to mean that they have been manipulated to either add at least one or more heterologous or exogenous nucleic acid residue and/or remove at least one endogenous (or native) nucleic acid residue. In some embodiments, the one or more nucleic acid residues that are added can be derived from an heterologous cell or the recombinant yeast host cell itself. In the latter scenario, the nucleic acid residue(s) is (are) added at a genomic location which is different than the native genomic location. The genetic manipulations did not occur in nature and are the results of in vitro manipulations of the native yeast host cell.

When expressed in a recombinant yeast host cell, the heterologous polypeptides (including the heterologous enzymes) described herein are encoded on one or more heterologous nucleic acid molecule. The term “heterologous” when used in reference to a nucleic acid molecule (such as a promoter or a coding sequence) or a polypeptide refers to a nucleic acid molecule/polypeptide that is not natively found in the recombinant host cell. “Heterologous” also includes a native coding region, or portion thereof, that was removed from the source organism and subsequently reintroduced into the source organism in a form that is different from the corresponding native gene, e.g., not in its natural location in the organism's genome. The heterologous nucleic acid molecule is purposively introduced into the recombinant host cell. The term “heterologous” as used herein also refers to an element (nucleic acid or polypeptide) that is derived from a source other than the endogenous source. Thus, for example, an heterologous element could be derived from a different strain of host cell, or from an organism of a different taxonomic group (e.g., different kingdom, phylum, class, order, family genus, or species, or any subgroup within one of these classifications). The term “heterologous” is also used synonymously herein with the term “exogenous”.

When an heterologous nucleic acid molecule is present in the recombinant yeast host cell, it can be integrated in the yeast host cell's genome. The term “integrated” as used herein refers to genetic elements that are placed, through molecular biology techniques, into the genome of a host cell. For example, genetic elements can be placed into the chromosomes of the host cell as opposed to in a vector such as a plasmid carried by the host cell. Methods for integrating genetic elements into the genome of a host cell are well known in the art and include homologous recombination. The heterologous nucleic acid molecule can be present in one or more copies in the yeast host cell's genome. Alternatively, the heterologous nucleic acid molecule can be independently replicating from the host cell's genome. In such embodiment, the nucleic acid molecule can be stable and self-replicating.

In some embodiments, heterologous nucleic acid molecules which can be introduced into the recombinant yeast host cells are codon-optimized with respect to the intended recipient recombinant yeast host cell. As used herein, the term “codon-optimized coding region” means a nucleic acid coding region that has been adapted for expression in the cells of a given organism by replacing at least one, or more than one, codons with one or more codons that are more frequently used in the genes of that organism. In general, highly expressed genes in an organism are biased towards codons that are recognized by the most abundant tRNA species in that organism. One measure of this bias is the “codon adaptation index” or “CAI,” which measures the extent to which the codons used to encode each amino acid in a particular gene are those which occur most frequently in a reference set of highly expressed genes from an organism. The CAI of codon optimized heterologous nucleic acid molecule described herein corresponds to between about 0.8 and 1.0, between about 0.8 and 0.9, or about 1.0.

The heterologous nucleic acid molecules of the present disclosure can comprise a coding region for the one or more heterologous polypeptides (including heterologous enzymes) to be expressed by the recombinant host cell and/or one or more regulatory regions. A DNA or RNA “coding region” is a DNA or RNA molecule which is transcribed and/or translated into a polypeptide in a cell in vitro or in vivo when placed under the control of appropriate regulatory sequences. “Regulatory regions” refer to nucleic acid regions located upstream (5′ non-coding sequences), within, or downstream (3 non-coding sequences) of a coding region, and which influence the transcription, RNA processing or stability, or translation of the associated coding region. Regulatory regions may include promoters, translation leader sequences, RNA processing sites, effector binding sites and stem-loop structures. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region. In an embodiment, the coding region can be referred to as an open reading frame. “Open reading frame” is abbreviated ORF and means a length of nucleic acid, either DNA, cDNA or RNA, that comprises a translation start signal or initiation codon, such as an ATG or AUG, and a termination codon and can be potentially translated into a polypeptide sequence.

The nucleic acid molecules described herein can comprise a non-coding region, for example a transcriptional and/or translational control regions. “Transcriptional and translational control regions” are DNA regulatory regions, such as promoters, enhancers, terminators, and the like, that provide for the expression of a coding region in a host cell. In eukaryotic cells, polyadenylation signals are control regions.

The heterologous nucleic acid molecule can be introduced and optionally maintained in the host cell using a vector. A “vector,” e.g., a “plasmid”, “cosmid” or “artificial chromosome” (such as, for example, a yeast artificial chromosome) refers to an extra chromosomal element and is usually in the form of a circular double-stranded DNA molecule. Such vectors may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a host cell.

In the heterologous nucleic acid molecules described herein, the promoters and the nucleic acid molecules coding for the one or more heterologous polypeptides (including heterologous enzymes) can be operatively linked to one another. In the context of the present disclosure, the expressions “operatively linked” or “operatively associated” refers to fact that the promoter is physically associated to the nucleotide acid molecule coding for the one or more heterologous polypeptide in a manner that allows, under certain conditions, for expression of the one or more heterologous polypeptide from the heterologous nucleic acid molecule. In an embodiment, the promoter can be located upstream (5′) of the nucleic acid sequence coding for the one or more heterologous polypeptide. In still another embodiment, the promoter can be located downstream (3′) of the nucleic acid sequence coding for the one or more heterologous polypeptide. In the context of the present disclosure, one or more than one promoter can be included in the heterologous nucleic acid molecule. When more than one promoter is included in the heterologous nucleic acid molecule, each of the promoters is operatively linked to the nucleic acid sequence coding for the one or more heterologous polypeptide. The promoters can be located, in view of the nucleic acid molecule coding for the one or more heterologous polypeptide, upstream, downstream as well as both upstream and downstream.

The expression “promoter” refers to a DNA fragment capable of controlling the expression of a coding sequence or functional RNA. The term “expression” as used herein, refers to the transcription and stable accumulation of sense (mRNA) from the heterologous nucleic acid molecule described herein. Expression may also refer to translation of mRNA into a polypeptide. Promoters may be derived in their entirety from a native gene, or be composed of different elements derived from different promoters found in nature, or even comprise synthetic DNA segments. It is understood by those skilled in the art that different promoters may direct the expression at different stages of development, or in response to different environmental or physiological conditions. Promoters which cause a gene to be expressed in most cells at most times at a substantial similar level are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as polypeptide binding domains (consensus sequences) responsible for the binding of the polymerase.

In the context of the present disclosure, the promoter controlling the expression of the heterologous polypeptide can be a constitutive promoter (such as, for example, tef2p (e.g., the promoter of the tef2 gene), cwp2p (e.g., the promoter of the cwp2 gene), ssa1p (e.g., the promoter of the ssa1 gene), eno1p (e.g., the promoter of the eno1 gene), hxk1 (e.g., the promoter of the hxk1 gene) and/or pgk1p (e.g., the promoter of the pgk1 gene). However, is some embodiments, it is preferable to limit the expression of the heterologous polypeptide. As such, the promoter controlling the expression of the heterologous polypeptide can be an inducible or modulated promoters such as, for example, a glucose-regulated promoter (e.g., the promoter of the hxt7 gene (referred to as hxt7p)) or a sulfite-regulated promoter (e.g., the promoter of the gpd2 gene (referred to as gpd2p or the promoter of the fzf1 gene (referred to as the fzf1p)), the promoter of the ssu1 gene (referred to as ssu1p), the promoter of the ssu1-r gene (referred to as ssur1-rp). In an embodiment, the promoter is an anaerobic-regulated promoters, such as, for example tdh1p (e.g., the promoter of the tdh1 gene), pau5p (e.g., the promoter of the pau5 gene), hor7p (e.g., the promoter of the hor7 gene), adh1p (e.g., the promoter of the adh1 gene), tdh2p (e.g., the promoter of the tdh2 gene), tdh3p (e.g., the promoter of the tdh3 gene), gpd1p (e.g., the promoter of the gdp1 gene), cdc19p (e.g., the promoter of the cdc19 gene), eno2p (e.g., the promoter of the eno2 gene), pdc1p (e.g., the promoter of the pdc1 gene), hxt3p (e.g., the promoter of the hxt3 gene), dan1 (e.g., the promoter of the dan1 gene) and tpi1p (e.g., the promoter of the tpi1 gene). In yet another embodiment, the promoter is a cytochrome c/mitochondrial electron transport chain promoter, such as, for example, the cyc1p (e.g., the promoter of the cyc1 gene) and/or the qcr8p (e.g., the promoter of the qcr8 gene). In an embodiment, the promoter used to allow the expression of the heterologous polypeptide is the adh1p. One or more promoters can be used to allow the expression of each heterologous polypeptides in the recombinant yeast host cell.

In embodiments in which the heterologous polypeptide has formate dehydrogenase activity uses NADP⁺ as a primary cofactor (such as, for example, the polypeptide the amino acid sequence of SEQ ID NO: 2, 3, 4, 21, 23, 25, 26 or 27, variants thereof and fragments thereof), the promoter used to allow its expression can be the tef2p, the ssa1p, the cdc19p, the tip1p, the cyc1p, the pgk1p, the tdh2p, the eno2p, the htx3p, the qcr8p, the tdh1p, the tdh3p and/or the hor7p. In a specific embodiment in which it is warranted to promote the use of NADP⁺ cofactor instead of the NAD⁺ cofactor, the promoter used to allow the expression of the heterologous polypeptide having formate dehydrogenase activity uses NADP⁺ as a primary cofactor, can be the pgk1p, the eno2p and/or the tdh2p.

One or more promoters can be used to allow the expression of each heterologous polypeptides in the recombinant yeast host cell. In the context of the present disclosure, the expression “functional fragment of a promoter” when used in combination to a promoter refers to a shorter nucleic acid sequence than the native promoter which retain the ability to control the expression of the nucleic acid sequence encoding the heterologous polypeptide. Usually, functional fragments are either 5′ and/or 3′ truncation of one or more nucleic acid residue from the native promoter nucleic acid sequence.

The promoter can be heterologous to the nucleic acid molecule encoding the one or more heterologous polypeptides. The promoter can be heterologous or derived from a strain being from the same genus or species as the recombinant yeast host cell. In an embodiment, the promoter is derived from the same genus or species of the yeast host cell and the heterologous polypeptide is derived from different genus that the host cell.

In an embodiment, the present disclosure concerns the expression of one or more polypeptides (including an enzyme), a variant thereof or a fragment thereof in a recombinant host cell. A variant comprises at least one amino acid difference when compared to the amino acid sequence of the native polypeptide and exhibits a biological activity substantially similar to the native polypeptide. The polypeptide “variants” have at least 50%, 55%, 60%, 65%, 70%, 75%, 80%, 85%, 90%, 95%, 96%, 97%, 98% or 99% of the biological activity of the wild-type heterologous polypeptide described herein. The polypeptide “variants” have at least 50%, 55%, 60%, 65%, 70%, 75%, 80%, 85%, 90%, 95%, 96%, 97%, 98% or 99% identity to the polypeptide described herein. The term “percent identity”, as known in the art, is a relationship between two or more polypeptide sequences or two or more polynucleotide sequences, as determined by comparing the sequences. The level of identity can be determined conventionally using known computer programs. Identity can be readily calculated by known methods, including but not limited to those described in: Computational Molecular Biology (Lesk, A. M., ed.) Oxford University Press, N Y (1988); Biocomputing: Informatics and Genome Projects (Smith, D. W., ed.) Academic Press, N Y (1993); Computer Analysis of Sequence Data, Part I (Griffin, A. M., and Griffin, H. G., eds.) Humana Press, N J (1994); Sequence Analysis in Molecular Biology (von Heinje, G., ed.) Academic Press (1987); and Sequence Analysis Primer (Gribskov, M. and Devereux, J., eds.) Stockton Press, NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignments of the sequences disclosed herein were performed using the Clustal method of alignment (Higgins and Sharp (1989) CABIOS. 5:151-153) with the default parameters (GAP PENALTY=10, GAP LENGTH PEN ALT Y=10). Default parameters for pairwise alignments using the Clustal method were KTUPLB 1, GAP PENALTY=3, WINDOW=5 and DIAGONALS SAVED=5.

The variant polypeptide described herein may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide for purification of the polypeptide.

A “variant” of the polypeptide can be a conservative variant or an allelic variant. As used herein, a conservative variant refers to alterations in the amino acid sequence that do not adversely affect the biological functions of the enzyme. A substitution, insertion or deletion is said to adversely affect the polypeptide when the altered sequence prevents or disrupts a biological function associated with the enzyme. For example, the overall charge, structure or hydrophobic-hydrophilic properties of the polypeptide can be altered without adversely affecting a biological activity. Accordingly, the amino acid sequence can be altered, for example to render the polypeptide more hydrophobic or hydrophilic, without adversely affecting the biological activities of the enzyme.

The heterologous polypeptide can be a fragment of the heterologous polypeptide or fragment of the variant heterologous polypeptide. A polypeptide fragment comprises at least one less amino acid residue when compared to the amino acid sequence of the native full-length polypeptide or polypeptide variant and possesses and still possess a biological activity substantially similar to the native full-length polypeptide or polypeptide variant. In some embodiments, the polypeptide “fragments” have at least 50%, 55%, 60%, 65%, 70%, 75%, 80%, 85%, 90%, 95%, 96%, 97%, 98% or 99% of the biological activity of the full-length polypeptides described herein. Polypeptide “fragments” have at least 100, 200, 300, 400, 500 or more consecutive amino acids of the heterologous polypeptide or the heterologous polypeptide variant. The polypeptide “fragments” have at least 50%, 55%, 60%, 65%, 70%, 75%, 80%, 85%, 90%, 95%, 96%, 97%, 98% or 99% identity to the full-length polypeptides described herein. In some embodiments, fragments of the polypeptides can be employed for producing the corresponding full-length polypeptide by peptide synthesis. Therefore, the fragments can be employed as intermediates for producing the full-length polypeptide.

In some additional embodiments, the present disclosure also provides expressing a polypeptide encoded by a gene ortholog of a gene known to encode the polypeptide. A “gene ortholog” is understood to be a gene in a different species that evolved from a common ancestral gene by speciation. In the context of the present disclosure, a gene ortholog encodes a polypeptide exhibiting a biological activity substantially similar to the native polypeptide.

In some further embodiments, the present disclosure also provides expressing a polypeptide encoded by a gene paralog of a gene known to encode the polypeptide A “gene paralog” is understood to be a gene related by duplication within the genome. In the context of the present disclosure, a gene paralog encodes a polypeptide that could exhibit additional biological functions when compared to the native polypeptide.

In some embodiments, the recombinant yeast host cell does include native formate dehydrogenase (FDH) genes and is capable of expressing native formate dehydrogenase genes (including orthologs and paralogs thereof). In yeasts, including S. cerevisiae, the native FDH genes include, without limitation, FDH1 and FDH2. As such, in some specific embodiments, the recombinant yeast host cell does include native FDH1 and FDH2 genes and is capable of expressing native FDH1 and FDH2 genes. Alternatively or in combination, the further yeast host cell does include native formate dehydrogenase (FDH) genes and is not capable of expressing native formate dehydrogenase genes (including orthologs and paralogs thereof). in some specific embodiments, the further yeast host cell does include native FDH1 and FDH2 genes and is capable of expressing native FDH1 and FDH2 genes.

In some alternative embodiments, the recombinant yeast host cell previously had native formate dehydrogenase (FDH) genes which have been inactivated. As such, the recombinant yeast host cell cannot include nor express native FDH genes (including orthologs and paralogs thereof), such as FDH1 and/or FDH2. In a specific embodiment, the recombinant yeast host cell has been modified to inactivate the native FDH1 and FDH2 genes. In some alternative embodiments, the further yeast host cell previously had native formate dehydrogenase (FDH) genes which have been inactivated. As such, the further yeast host cell cannot include nor express native FDH genes (including orthologs and paralogs thereof), such as FDH1 and/or FDH2. In a specific embodiment, the further yeast host cell has been modified to inactivate the native FDH1 and FDH2 genes.

In the context of the present disclosure, the expression “formate dehydrogenase” refers to an enzyme capable of catalyzing the conversion of formate into carbon dioxide (E.C. 1.2.1.2). This catalysis also involves the use of a cofactor, NAD⁺ or NADP⁺, and its conversion into NAPH or NADPH. The formate dehydrogenases of the present disclosure do include enzymes which uses NAD⁺ or NADP⁺ as a primary cofactor. In Saccharomyces cerevisiae, there are at least two genes encoding FDH: FDH1 (also known as YOR388C and having the SGD ID: SGD:S000005915) and FDH2 (also known YPL275W and having the SGC ID: SGD:S000006196). As such, when the recombinant yeast host cell and/or the further yeast host cell is from the species Saccharomyces cerevisiae, it is contemplated that the yeast host cell has at least one or both native FDH genes and expresses at least one or both FDH genes. Alternatively, when the recombinant yeast host cell and/or the further yeast host cell is from the species Saccharomyces cerevisiae, it is contemplated that the yeast host cell previously had at least one or both native FDH genes and that at least one or both FDH genes have been inactivated in such a way that the yeast host cell fails to express at least one or both native FDH genes. In a specific embodiment, the recombinant yeast host cell includes genetic modifications in its native FDH genes which prevent the expression of the native FDH genes.

In the context of the present disclosure, the recombinant/native/further yeast host cell is a yeast. Suitable yeast host cells can be, for example, from the genus Saccharomyces, Kluyveromyces, Arxula, Debaryomyces, Candida, Pichia, Phaffia, Schizosaccharomyces, Hansenula, Kloeckera, Schwanniomyces or Yarrowia. Suitable yeast species can include, for example, S. cerevisiae, S. bulderi, S. barnetti, S. exiguus, S. uvarum, S. diastaticus, K. lactis, K. marxianus or K. fragilis. In some embodiments, the yeast is selected from the group consisting of Saccharomyces cerevisiae, Schizzosaccharomyces pombe, Candida albicans, Pichia pastoris, Pichia stipitis, Yarrowia lipolytica, Hansenula polymorpha, Phaffia rhodozyma, Candida utilis, Arxula adeninivorans, Debaryomyces hansenii, Debaryomyces polymorphus, Schizosaccharomyces pombe and Schwanniomyces occidentalis. In one particular embodiment, the yeast is Saccharomyces cerevisiae. In some embodiments, the host cell can be an oleaginous yeast cell. For example, the oleaginous yeast host cell can be from the genus Blakeslea, Candida, Cryptococcus, Cunninghamella, Lipomyces, Mortierella, Mucor, Phycomyces, Pythium, Rhodosporidum, Rhodotorula, Trichosporon or Yarrowia. In some alternative embodiments, the recombinant/native/further yeast host cell can be an oleaginous microalgae host cell (e.g., for example, from the genus Thraustochytrium or Schizochytrium). In an embodiment, the recombinant/native/further yeast host cell is from the genus Saccharomyces and, in some additional embodiments, from the species Saccharomyces cerevisiae.

Since the recombinant yeast host cell can be used for the fermentation of a biomass and the generation of fermentation product, it is contemplated herein that it has the ability (or has been genetically modified to have the ability) to convert a biomass into a fermentation product without the including the first, second and/or third genetic modifications described herein. In some embodiments, the parental strain used to make the recombinant yeast host cell of the present disclosure has the ability (or has been genetically modified to have the ability) to convert a biomass into a fermentation product and has been modified to include the at least first genetic modification (and optionally the second genetic modification) to generate the recombinant yeast host cell. In an embodiment, the recombinant yeast host cell (or its corresponding parental strain) has the ability to convert starch into ethanol during fermentation, as it is described below.

First Genetic Modification for Increasing Formate Production

In the present disclosure, the recombinant yeast host cell does include a first genetic modification for increasing the fermentation yield which results in formate production/accumulation (internally and/or externally in the fermentation medium). The first genetic modification is done purposefully to increase formate production/accumulation, to ultimately increase the production/accumulation of a metabolic product useful for increasing fermentation yield. This metabolic product can be, without limitation, acetyl-CoA. This increase in formate production is relative to a corresponding native yeast host cell (such as for example a parental yeast strain) which does not include the first genetic modification (and in some embodiments, is otherwise genetically identical to the recombinant yeast host cell). In some embodiments, especially when the recombinant yeast host cell is used in the production of a biofuel, this increase in formate production is also associated with an increase in the production of acetyl-CoA when compared to the corresponding native yeast host cell.

The increased in formate production is due at least in part to the introduction of one or more first genetic modification(s) in a native or parental yeast host cell to obtain the recombinant yeast host cell. For example, the first genetic modification can be done to the transcriptional regulatory elements of one or more genes encoding a polypeptide capable of making formate. In yet another example, the first genetic modification can be done to reduce the expression or inactivate an inhibitor of the polypeptide capable of making formate. Alternatively or in combination, the first genetic modification can include adding a first heterologous nucleic acid encoding a first heterologous polypeptide capable of making formate in the recombinant yeast host cell. The present disclosure thus provides a recombinant yeast host cell comprising a first heterologous nucleic acid molecule encoding a first heterologous polypeptide capable of making formate in the recombinant yeast host cell. As such, the activity of the one or more first heterologous polypeptides capable of making formate of the recombinant yeast host cell is considered “increased” because it is higher than the activity associated with the native yeast host cell (e.g., prior to the introduction of the one or more first genetic modifications). The one or more first genetic modifications is not limited to a specific modification provided that it does increase the activity, and in some embodiments, the expression of the one or more pyruvate formate lyase or PFL polypeptides.

In an embodiment, the first genetic modification does achieve higher pyruvate formate lyase activity in the recombinant yeast host cell. This increase in pyruvate formate lyase activity is relative to a corresponding native yeast host cell which does not include the first genetic modification. As used in the context of the present disclosure, the term “pyruvate formate lyase” or “PFL” refers to an enzyme (EC 2.3.1.54) also known as formate C-acetyltransferase, pyruvate formate-lyase, pyruvic formate-lyase and formate acetyltransferase. Pyruvate formate lyases are capable of catalyzing the conversion of coenzyme A (CoA) and pyruvate into acetyl-CoA and formate. In some embodiments, the pyruvate formate lyase activity may be increased by expressing an heterologous pyruvate formate lyase activating enzyme and/or a pyruvate formate lyase enzymate (such as, for example PFLA and/or PFLB).

In the context of the present disclosure, the first genetic modification can include the introduction of an heterologous nucleic acid molecule encoding a pyruvate formate lyase activating enzyme and/or a puryvate formate lyase enzyme, such as PFLA. Embodiments of the pyruvate formate lyase activating enzyme and of PFLA can be derived, without limitation, from the following (the number in brackets correspond to the Gene ID number): Escherichia coli (MG1655945517), Shewanella oneidensis (1706020), Bifidobacterium longum (1022452), Mycobacterium bovis (32287203), Haemophilus parasuis (7277998), Mannheimia haemolytica (15341817), Vibrio vulnificus (33955434), Cronobacter sakazakii (29456271), Vibrio alginolyticus (31649536), Pasteurella multocida (29388611), Aggregatibacter actinomycetemcomitans (31673701), Actinobacillus suis (34291363), Finegoldia magna (34165045), Zymomonas mobilis subsp. mobilis (3073423), Vibrio tubiashii (23444968), Gallibacterium anatis (10563639), Actinobacillus pleuropneumoniae serovar (4849949), Ruminiclostridium thermocellum (35805539), Cylindrospermopsis raciborskii (34474378), Lactococcus garvieae (34204939), Bacillus cytotoxicus (33895780), Providencia stuartii (31518098), Pantoea ananatis (31510290), Teredinibacter turnerae (29648846), Morganella morganii subsp. morganii (14670737), Vibrio anguillarum (77510775106), Dickeya dadantii (39379733484), Xenorhabdus bovienii (8830449), Edwardsiella ictaluri (7959196), Proteus mirabilis (6801040), Rahnella aquatilis (34350771), Bacillus pseudomycoides (34214771), Vibrio alginolyticus (29867350), Vibrio nigripulchritudo (29462895), Vibrio orientalis (25689084), Kosakonia sacchari (23844195), Serratia marcescens subsp. marcescens (23387394), Shewanella baltica (11772864), Vibrio vulnificus (2625152), Streptomyces acidiscabies (33082227), Streptomyces davaonensis (31227069), Streptomyces scabiei (24308152), Volvox carteri f. nagariensis (9616877), Vibrio breoganii (35839746), Vibrio mediterranei (34766273), Fibrobacter succinogenes subsp. succinogenes (34755395), Enterococcus gilvus (34360882), Akkermansia muciniphila (34173806), Enterobacter hormaechei subsp. Steigerwaltii (34153767), Dickeya zeae (33924935), Enterobacter sp. (32442159), Serratia odorifera (31794665), Vibrio crassostreae (31641425), Selenomonas ruminantium subsp. lactilytica (31522409), Fusobacterium necrophorum subsp. funduliforme (31520833), Bacteroides uniformis (31507008), Haemophilus somnus (233631487328), Rodentibacter pneumotropicus (31211548), Pectobacterium carotovorum subsp. carotovorum (29706463), Eikenella corrodens (29689753), Bacillus thuringiensis (29685036), Streptomyces rimosus subsp. Rimosus (29531909), Vibrio fluvialis (29387180), Klebsiella oxytoca (29377541), Parageobacillus thermoglucosidans (29237437), Aeromonas veronii (28678409), Clostridium innocuum (26150741), Neisseria mucosa (25047077), Citrobacter freundii (23337507), Clostridium bolteae (23114831), Vibrio tasmaniensis (7160642), Aeromonas salmonicida subsp. salmonicida (4995006), Escherichia coli 0157:H7 str. Sakai (917728), Escherichia coli 083:H1 str. (12877392), Yersinia pestis (11742220), Clostridioides difficile (4915332), Vibrio fischeri (3278678), Vibrio parahaemolyticus (1188496), Vibrio coralliilyticus (29561946), Kosakonia cowanii (35808238), Yersinia ruckeri (29469535), Gardnerella vaginalis (99041930), Listeria fleischmannii subsp. Coloradonensis (34329629), Photobacterium kishitanii (31588205), Aggregatibacter actinomycetemcomitans (29932581), Bacteroides caccae (36116123), Vibrio toranzoniae (34373279), Providencia alcalifaciens (34346411), Edwardsiella anguillarum (33937991), Lonsdalea quercina subsp. Quercina (33074607), Pantoea septica (32455521), Butyrivibrio proteoclasticus (31781353), Photorhabdus temperata subsp. Thracensis (29598129), Dickeya solani (23246485), Aeromonas hydrophila subsp. hydrophila (4489195), Vibrio cholerae 01 biovar El Tor str. (2613623), Serratia rubidaea (32372861), Vibrio bivalvicida (32079218), Serratia liquefaciens (29904481), Gilliamella apicola (29851437), Pluralibacter gergoviae (29488654), Escherichia coli 0104:H4 (13701423), Enterobacter aerogenes (10793245), Escherichia coli (7152373), Vibrio campbellii (5555486), Shigella dysenteriae (3795967), Bacillus thuringiensis serovar konkukian (2854507), Salmonella enterica subsp. enterica serovar Typhimurium (1252488), Bacillus anthracis (1087733), Shigella flexneri (1023839), Streptomyces griseoruber (32320335), Ruminococcus gnavus (35895414), Aeromonas fluvialis (35843699), Streptomyces ossamyceticus (35815915), Xenorhabdus doucetiae (34866557), Lactococcus piscium (34864314), Bacillus glycinifermentans (34773640), Photobacterium damselae subsp. Damselae 34509297, Streptomyces venezuelae 34035779, Shewanella algae (34011413), Neisseria sicca (33952518), Chania multitudinisentens (32575347), Kitasatospora purpeofusca (32375714), Serratia fonticola (32345867), Aeromonas enteropelogenes (32325051), Micromonospora aurantiaca (32162988), Moritella viscosa (31933483), Yersinia aldovae (31912331), Leclercia adecarboxylata (31868528), Salinivibrio costicola subsp. costicola (31850688), Aggregatibacter aphrophilus (31611082), Photobacterium leiognathi (31590325), Streptomyces canus (31293262), Pantoea dispersa (29923491), Pantoea rwandensis (29806428), Paenibacillus borealis (29548601), Aliivibrio wodanis (28541257), Streptomyces virginiae (23221817), Escherichia coli (7158493), Mycobacterium tuberculosis (887973), Streptococcus mutans (1028925), Streptococcus cristatus (29901602), Enterococcus hirae (13176624), Bacillus licheniformis (3031413), Chromobacterium violaceum (24949178), Parabacteroides distasonis (5308542), Bacteroides vulgatus (5303840), Faecalibacterium prausnitzii (34753201), Melissococcus plutonius (34410474), Streptococcus gallolyticus subsp. gallolyticus (34397064), Enterococcus malodoratus (34355146), Bacteroides oleiciplenus (32503668), Listeria monocytogenes (985766), Enterococcus faecalis (1200510), Campylobacter jejuni subsp. jejuni (905864), Lactobacillus plantarum (1063963), Yersinia enterocolitica subsp. enterocolitica (4713333), Streptococcus equinus (33961143), Macrococcus canis (35294771), Streptococcus sanguinis (4807186), Lactobacillus salivarius (3978441), Lactococcus lactis subsp. lactis (1115478), Enterococcus faecium (12999835), Clostridium botulinum A (5184387), Clostridium acetobutylicum (1117164), Bacillus thuringiensis serovar konkukian (2857050), Cryobacterium flavum (35899117), Enterovibrio norvegicus (35871749), Bacillus acidiceler (34874556), Prevotella intermedia (34516987), Pseudobutyrivibrio ruminis (34419801), Pseudovibrio ascidiaceicola (34149433), Corynebacterium coyleae (34026109), Lactobacillus curvatus (33994172), Cellulosimicrobium cellulans (33980622), Lactobacillus agilis (33975995), Lactobacillus sakei (33973512), Staphylococcus simulans (32051953), Obesumbacterium proteus (29501324), Salmonella enterica subsp. enterica serovar Typhi (1247402), Streptococcus agalactiae (1014207), Streptococcus agalactiae (1013114), Legionella pneumophila subsp. pneumophila str. Philadelphia (119832735), Pyrococcus furiosus (1468475), Mannheimia haemolytica (15340992), Thalassiosira pseudonana (7444511), Thalassiosira pseudonana (7444510), Streptococcus thermophilus (31940129), Sulfolobus solfataricus (1454925), Streptococcus iniae (35765828), Streptococcus iniae (35764800), Bifidobacterium thermophilum (31839084), Bifidobacterium animalis subsp. lactis (29695452), Streptobacillus moniliformis (29673299), Thermogladius calderae (13013001), Streptococcus oralis subsp. tigurinus (31538096), Lactobacillus ruminis (29802671), Streptococcus parauberis (29752557), Bacteroides ovatus (29454036), Streptococcus gordonii str. Challis substr. CH1 (25052319), Clostridium botulinum B str. Eklund 17B (19963260), Thermococcus litoralis (16548368), Archaeoglobus sulfaticallidus (15392443), Ferroglobus placidus (8778929), Archaeoglobus profundus (8739370), Listeria seeligeri serovar 1/2b (32488230), Bacillus thuringiensis (31632063), Rhodobacter capsulatus (31491679), Clostridium botulinum (29749009), Clostridium perfringens (29571530), Lactococcus garvieae (12478921), Proteus mirabilis (6799920), Lactobacillus animalis (32012274), Vibrio alginolyticus (29869205), Bacteroides thetaiotaomicron (31617701), Bacteroides thetaiotaomicron (31617140), Bacteroides cellulosilyticus (29608790), Bacteroides ovatus (29453452), Bacillus mycoides (29402181), Chlamydomonas reinhardtii (5726206), Fusobacterium periodonticum (35833538), Selenomonas flueggei (32477557), Selenomonas noxia (32475880), Anaerococcus hydrogenalis (32462628), Centipeda periodontii (32173931), Centipeda periodontii (32173899), Streptococcus thermophilus (31938326), Enterococcus durans (31916360), Fusobacterium nucleatum (31730399), Anaerostipes hadrus (31625694), Anaerostipes hadrus (31623667), Enterococcus haemoperoxidus (29838940), Gardnerella vaginalis (29692621), Streptococcus salivarius (29397526), Klebsiella oxytoca (29379245), Bifidobacterium breve (29241363), Actinomyces odontolyticus (25045153), Haemophilus ducreyi (24944624), Archaeoglobus fulgidus (24793671), Streptococcus uberis (24161511), Fusobacterium nucleatum subsp. animalis (23369066), Corynebacterium accolens (23249616), Archaeoglobus veneficus (10394332), Prevotella melaninogenica (9497682), Aeromonas salmonicida subsp. salmonicida (4997325), Pyrobaculum islandicum (4616932), Thermofilum pendens (4600420), Bifidobacterium adolescentis (4556560), Listeria monocytogenes (986485), Bifidobacterium thermophilum (35776852), Methanothermobacter sp. CaT2 (24854111), Streptococcus pyogenes (901706), Exiguobacterium sibiricum (31768748), Clostridioides difficile (4916015), Clostridioides difficile (4913022), Vibrio parahaemolyticus (1192264), Yersinia enterocolitica subsp. enterocolitica (4712948), Enterococcus cecorum (29475065), Bifidobacterium pseudolongum (34879480), Methanothermus fervidus (9962832), Methanothermus fervidus (9962056), Corynebacterium simulans (29536891), Thermoproteus uzoniensis (10359872), Vulcanisaeta distributa (9752274), Streptococcus mitis (8799048), Ferroglobus placidus (8778420), Streptococcus suis (8153745), Clostridium novyi (4541619), Streptococcus mutans (1029528), Thermosynechococcus elongatus (1010568), Chlorobium tepidum (1007539), Fusobacterium nucleatum subsp. nucleatum (993139), Streptococcus pneumoniae (933787), Clostridium baratii (31579258), Enterococcus mundtii (31547246), Prevotella ruminicola (31500814), Aeromonas hydrophila subsp. hydrophila (4490168), Aeromonas hydrophila subsp. hydrophila (4487541), Clostridium acetobutylicum (1117604), Chromobacterium subtsugae (31604683), Gilliamella apicola (29849369), Klebsiella pneumoniae subsp. pneumoniae (11846825), Enterobacter cloacae subsp. cloacae (9125235), Escherichia coli (7150298), Salmonella enterica subsp. enterica serovar Typhimurium (1252363), Salmonella enterica subsp. enterica serovar Typhi (1247322), Bacillus cereus (1202845), Bacteroides thetaiotaomicron (1074343), Bacteroides thetaiotaomicron (1071815), Bacillus coagulans (29814250), Bacteroides cellulosilyticus (29610027), Bacillus anthracis (2850719), Monoraphidium neglectum (25735215), Monoraphidium neglectum (25727595), Alloscardovia omnicolens (35868062), Actinomyces neuii subsp. neuii (35867196), Acetoanaerobium sticklandii (35557713), Exiguobacterium undae (32084128), Paenibacillus pabuli (32034589), Paenibacillus etheri (32019864), Actinomyces oris (31655321), Vibrio alginolyticus (31651465), Brochothrix thermosphacta (29820407), Lactobacillus sakei subsp. sakei (29638315), Anoxybacillus gonensis (29574914), variants thereof as well as fragments thereof. In an embodiment, the PFLA polypeptide is derived from the genus Bifidobacterium sp. and in some embodiments from the species Bifidobacterium adolescentis. In such embodiments, the PFLA polypeptide can have the amino acid sequence of SEQ ID NO: 6, be a variant of SEQ ID NO: 6 or be a fragment of SEQ ID NO: 6. In another embodiment, the recombinant yeast host cell comprises a nucleic acid molecule having the nucleic acid sequence of SEQ ID NO: 14 or 15. In an embodiment, the heterologous nucleic acid molecule encoding the PFLA polypeptide is present in at least one, two, three, four, five or more copies in the recombinant yeast host cell. In still another embodiment, the heterologous nucleic acid molecule encoding the PFLA polypeptide is present in no more than five, four, three, two or one copy/ies in the recombinant yeast host cell.

In the context of the present disclosure, the first genetic modification can include the introduction of an heterologous nucleic acid molecule encoding a formate acetyltransferase enzyme and/or a puryvate formate lyase enzyme, such as PFLB. Embodiments of PFLB can be derived, without limitation, from the following (the number in brackets correspond to the Gene ID number): Escherichia coli (945514), Shewanella oneidensis (1170601), Actinobacillus suis (34292499), Finegoldia magna (34165044), Streptococcus cristatus (29901775), Enterococcus hirae (13176625), Bacillus (3031414), Providencia alcalifaciens (34345353), Lactococcus garvieae (34203444), Butyrivibrio proteoclasticus (31781354), Teredinibacter turnerae (29651613), Chromobacterium violaceum (24945652), Vibrio campbellii (5554880), Vibrio campbellii (5554796), Rahnella aquatilis HX2 (34351700), Serratia rubidaea (32375076), Kosakonia sacchari SP1 (23845740), Shewanella baltica (11772863), Streptomyces acidiscabies (33082309), Streptomyces davaonensis (31227068), Parabacteroides distasonis (5308541), Bacteroides vulgatus (5303841), Fibrobacter succinogenes subsp. succinogenes (34755392), Photobacterium damselae subsp. Damselae (34512678), Enterococcus gilvus (34361749), Enterococcus gilvus (34360863), Enterococcus malodoratus (34355213), Enterococcus malodoratus (34354022), Akkermansia muciniphila (34174913), Lactobacillus curvatus (33995135), Dickeya zeae (33924934), Bacteroides oleiciplenus (32502326), Micromonospora aurantiaca (32162989), Selenomonas ruminantium subsp. lactilytica (31522408), Fusobacterium necrophorum subsp. funduliforme (31520832), Bacteroides uniformis (31507007), Streptomyces rimosus subsp. Rimosus (29531908), Clostridium innocuum (26150740), Haemophilus] ducreyi (24944556), Clostridium bolteae (23114829), Vibrio tasmaniensis (7160644), Aeromonas salmonicida subsp. salmonicida (4997718), Listeria monocytogenes (986171), Enterococcus faecalis (1200511), Lactobacillus plantarum (1064019), Vibrio fischeri (3278780), Lactobacillus sakei (33973511), Gardnerella vaginalis (9904192), Vibrio vulnificus (33954428), Vibrio toranzoniae (34373229), Anaerostipes hadrus (34240161), Edwardsiella anguillarum (33940299), Edwardsiella anguillarum (33937990), Lonsdalea quercina subsp. Quercina (33074710), Enterococcus faecium (12999834), Aeromonas hydrophila subsp. hydrophila (4489100), Clostridium acetobutylicum (1117163), Escherichia coli (7151395), Shigella dysenteriae (3795966), Bacillus thuringiensis serovar konkukian (2856201), Salmonella enterica subsp. enterica serovar Typhimurium (1252491), Shigella flexneri (1023824), Streptomyces griseoruber (32320336), Cryobacterium flavum (35898977), Ruminococcus gnavus (35895748), Bacillus acidiceler (34874555), Lactococcus piscium (34864362), Vibrio mediterranei (34766270), Faecalibacterium prausnitzii (34753200), Prevotella intermedia (34516966), Photobacterium damselae subsp. Damselae (34509286), Pseudobutyrivibrio ruminis (34419894), Melissococcus plutonius (34408953), Streptococcus gallolyticus subsp. gallolyticus (34398704), Enterobacter hormaechei subsp. Steigerwaltii (34155981), Enterobacter hormaechei subsp. Steigerwaltii (34152298), Streptomyces venezuelae (34036549), Shewanella algae (34009243), Lactobacillus agilis (33976013), Streptococcus equinus (33961013), Neisseria sicca (33952517), Kitasatospora purpeofusca (32375782), Paenibacillus borealis (29549449), Vibrio fluvialis (29387150), Aliivibrio wodanis (28542465), Aliivibrio wodanis (28541256), Escherichia coli (7157421), Salmonella enterica subsp. enterica serovar Typhi (1247405), Yersinia pestis (1174224), Yersinia enterocolitica subsp. enterocolitica (4713334), Streptococcus suis (8155093), Escherichia coli (947854), Escherichia coli (946315), Escherichia coli (945513), Escherichia coli (948904), Escherichia coli (917731), Yersinia enterocolitica subsp. enterocolitica (4714349), variants thereof as well as fragments thereof. In an embodiment, the PFLB polypeptide is derived from the genus Bifidobacterium and in some embodiments from the specifies Bifidobacterium adolescentis.

In such embodiments, the PFLB polypeptide can have the amino acid sequence of SEQ ID NO: 7, be a variant of SEQ ID NO: 7 or be a fragment of SEQ ID NO: 7. In another embodiment, the recombinant yeast host cell comprises a nucleic acid molecule having the nucleic acid sequence of SEQ ID NO: 16 or 17. In an embodiment, the heterologous nucleic acid molecule encoding the PFLB polypeptide is present in at least one, two, three, four, five or more copies in the recombinant yeast host cell. In still another embodiment, the heterologous nucleic acid molecule encoding the PFLB polypeptide is present in no more than five, four, three, two or one copy/ies in the recombinant yeast host cell.

In some embodiments, the recombinant yeast host cell comprises a first genetic modification for expressing a PFLA polypeptide, a PFLB polypeptide or a combination. In a specific embodiment, the recombinant yeast host cell comprises a first genetic modification for expressing a PFLA polypeptide and a PFLB polypeptide which can, in some embodiments, be provided on distinct heterologous nucleic acid molecules. As indicated below, the recombinant yeast host cell can also include additional genetic modifications to provide or increase its ability to transform acetyl-CoA into an alcohol such as ethanol.

Source of Formate Dehydrogenase Activity

The recombinant yeast host cell of the present disclosure is provided with a source of formate dehydrogenase (FDH) activity. FDH activity can be provided from an external source (another microorganism such as, for example, a further yeast host cell). Alternatively or in combination, FDH activity can be provided from an internal source (by increasing the FDH activity of the recombinant yeast host cell). In both of these embodiments, the recombinant yeast host cell and/or the further yeast host cell can bear and express at least one or both native FDH genes, orthologs thereof and paralogs thereof. Alternatively, still in both of these embodiments, the recombinant yeast host cell and/or the further yeast host cell can include a second genetic modification aimed at inactivating at least one or both FDH native gene(s), orthologs thereof or paralogs thereof. In still a further embodiment, the recombinant yeast host cell and/or the further yeast host cell can include a fourth and/or fifth genetic modification(s) aimed at inactivating both FDH native gene(s), orthologs thereof or paralogs thereof. The inactivation of a native FDH gene can be done, for example, by deleting at least one nucleic acid residue from the non-coding or coding sequence of the native FDH gene so as to limit or inhibit the expression of the gene and/or to disrupt open reading frame or remove the coding sequence of the native FDH gene(s).

In an embodiment, the source of formate dehydrogenase activity is internal and is provided by introducing a second genetic modification in the recombinant yeast host cell aimed at increasing the FDH activity in the cell. For example, the second genetic modification can be done to the transcriptional regulatory elements of one or more genes encoding a polypeptide having FDH activity. In yet another example, the second genetic modification can be done to limit the expression or inactivate an inhibitor of the polypeptide having FDH activity. Alternatively or in combination, the second genetic modification can include adding a second heterologous nucleic acid molecule encoding an heterologous polypeptide having FDH activity in the recombinant yeast host cell. The present disclosure thus provides a recombinant yeast host cell comprising a second heterologous nucleic acid molecule encoding an heterologous polypeptide having FDH activity. For example, the second genetic modification can include adding a second heterologous nucleic acid molecule encoding an heterologous FDH1 polypeptide. As such, the activity of the polypeptides having FDH activity of the recombinant yeast host cell is considered “increased” because it is higher than the activity of native yeast host cell (e.g., prior to the introduction of the one or more second genetic modifications). The second genetic modifications is not limited to a specific modification provided that it does increase the activity, and in some embodiments, the expression of the polypeptides having FDH activity. In a specific embodiment, the recombinant yeast host cell includes the second and fourth genetic modifications. In yet another embodiment, the recombinant yeast host cell includes the second genetic modification and does not include the fourth genetic modification.

In an embodiment, the source of formate dehydrogenase activity is external and is provided by a further yeast host cell exhibiting FDH activity (via the expression of its native FDH polypeptides) and/or by introducing a third genetic modification in the further yeast host cell (having or lacking native FDH activity) aimed at increasing the FDH activity in the cell. For example, the third genetic modification can be done to the transcriptional regulatory elements of one or more genes encoding a polypeptide having FDH activity. In yet another example, the third genetic modification can be done to limit the expression or inactivate an inhibitor of the polypeptide having FDH activity. Alternatively or in combination, the third genetic modification can include adding a third heterologous nucleic acid encoding an heterologous polypeptide having FDH activity in the further yeast host cell. Thus, the present disclosure provides a further yeast host cell comprising a third heterologous nucleic acid encoding an heterologous polypeptide having FDH activity. For example, the third genetic modification can include adding a third heterologous nucleic acid encoding an heterologous FDH1 polypeptide in the further yeast host cell. As such, the activity of the polypeptides having FDH activity of the further yeast host cell is considered “increased” because it is higher than the activity of native yeast host cell (e.g., prior to the introduction of the one or more third genetic modifications). The third genetic modifications is not limited to a specific modification provided that it does increase FDH activity in the further yeast host cell, and in some embodiments, the expression of the polypeptides having FDH activity in the further yeast host cell. In an embodiment, the further yeast host cell includes the third genetic modification, but not the fifth genetic modification.

As indicated above, the expression “formate dehydrogenase” refers to an enzyme capable of catalyzing the conversion of formate into carbon dioxide (E.C. 1.2.1.2). The expression “cell/polypeptide having FDH activity” refers to a cell expressing a polypeptide exhibiting FDH activity. The reaction catalyzed by the FDH polypeptide also involves the use of a cofactor, NAD⁺ or NADP⁺, and its conversion into NAPH or NADPH. The formate dehydrogenases of the present disclosure do include enzymes which uses NAD⁺ or NADP⁺ as the primary cofactor. A polypeptide having FDH activity and using NAD⁺ as a primary cofactor is a polypeptide which preferably uses NAD⁺ as its cofactor instead of NADP⁺ to perform its enzymatic activity. By the same token, a polypeptide having FDH activity and using NADP⁺ as a primary cofactor is a polypeptide which selectively uses NADP⁺ as its cofactor instead of NAD⁺ to perform its enzymatic activity. Polypeptides using NAD⁺ as a primary cofactor include, without limitations, those having the amino acid sequence of SEQ ID NO: 1 or 5, being variants of the amino acid sequence of SEQ ID NO: 1 or 5 or being fragments of the amino acid sequence of SEQ ID NO: 1 or 5. Polypeptides using NADP⁺ as a primary cofactor include, without limitations, those having the amino acid sequence of SEQ ID NO: 2, 3, 4, 21, 23, 25, 26 or 27 being variants of the amino acid sequence of SEQ ID NO: 2, 3, 4, 21, 23, 25, 26 or 27 or being fragments of the amino acid sequence of SEQ ID NO: 2, 3, 4, 21, 23, 25, 26 or 27. In still another embodiment, the polypeptide having FDH activity is from the genus Saccharomyces sp., for example Saccharomyces cerevisiae, and can be, in some additional embodiment, the FDH1 polypeptide. In yet another embodiment, the polypeptide having FDH activity is from the genus Lactobacillus sp., for example Lactobacillus buchneri.

In an embodiment, it is possible to change the FDH's primary cofactor by modifying the amino acid sequence of the polypeptide having FDH activity. As indicated in the publication of Serov et al. (2002), it is possible to modify the cofactor specificity from NAD⁺ to NADP⁺ of a polypeptide having FDH activity by introducing two single point mutations (D196A and Y197R). As also indicated in the publication of Wu et al. (2009). it is possible to modify the cofactor specificity from NAD⁺ to NADP⁺ of a polypeptide having FDH activity by introducing two or three single point mutations (at positions 195, 196 and/or 197, such as D195Q/Y196R and D195S/Y196P). As such, it is possible to provide a mutated polypeptide having FDH activity which uses NADP⁺ as a cofactor by introducing one or more point mutations as taught by Serov and/or Wu.

In Saccharomyces cerevisiae, there are at least two genes encoding FDH: FDH1 (also known as YOR388C and having the SGD ID: SGD:S000005915) and FDH2 (also known YPL275W and having the SGC ID: SGD:S000006196). Polypeptides having FDH activity can be derived from the following (the number in brackets correspond to the Gene ID number): Saccharomyces cerevisiae (854570), Zea mays (542459), Chlamydomonas reinhardtii (5719540), Candida albicans (3646398), Candida dubliniensis (8049981), Scheffersomyces stipitis (4851979), Trichoderma reesei (18483115), Aspergillus thermomutatus (38122179), Pseudogymnoascus destructans (36287283), Sugiyamaella lignohabitans (30037648), Sugiyamaella lignohabitans (30037647), Sugiyamaella lignohabitans (30035306), Sugiyamaella lignohabitans (30035195), Sugiyamaella lignohabitans (30033393), Solanum tuberosum (102577429), Capsicum annuum (107860635), Nicotiana attenuata (109206919), Candida orthopsilosis (14540065), Scheffersomyces stipitis (4840932), Scheffersomyces stipitis (4840931), Candida viswanathii (38108764), Candida viswanathii (38108751), Candida viswanathii (38107180), Candida viswanathii (38107168), Candida viswanathii (38107128), Candida viswanathii (38106332), Candida viswanathii (38101224), Candida viswanathii (38100400), Candida viswanathii (38100391), Saccharomyces cerevisiae (852241), Saccharomyces cerevisiae (852532), Candida dubliniensis (8050169), Candida dubliniensis (8048235), Saccharomyces cerevisiae (855853), Scheffersomyces stipitis (4837984), Saccharomyces cerevisiae (2827705), Zea mays (542657), Lactobacillus buchneri (34323951), variants thereof and fragments thereof. In an embodiment, the polypeptide having FDH activity has the amino acid sequence of SEQ ID NO: 1 (e.g., FDH1), is a variant of the amino acid sequence of SEQ ID NO: 1 or is a fragment of the amino acid sequence of SEQ ID NO: 1. In embodiments in which the polypeptide has FDH activity having the amino acid sequence of SEQ ID NO: 1, is a variant of the amino acid sequence of SEQ ID NO: 1 or is a fragment of the amino acid sequence of SEQ ID NO: 1, the recombinant yeast host cell expressing such polypeptide can include native FDH genes. In embodiments in which the polypeptide has FDH activity having the amino acid sequence of SEQ ID NO: 1, is a variant of the amino acid sequence of SEQ ID NO: 1 or is a fragment of the amino acid sequence of SEQ ID NO: 1, the recombinant yeast host cell expressing such polypeptide can has one or both native FDH genes inactivated. In another embodiment, the polypeptide having FDH activity has the amino acid sequence of SEQ ID NO: 2, is a variant of the amino acid sequence of SEQ ID NO: 2 or is a fragment of the amino acid sequence of SEQ ID NO: 2. In an embodiment, the polypeptide having FDH activity has the amino acid sequence of SEQ ID NO: 3, is a variant of the amino acid sequence of SEQ ID NO: 3 or is a fragment of the amino acid sequence of SEQ ID NO: 3. In an embodiment, the polypeptide having FDH activity has the amino acid sequence of SEQ ID NO: 4, is a variant of the amino acid sequence of SEQ ID NO: 4 or is a fragment of the amino acid sequence of SEQ ID NO: 4.

In an embodiment, the polypeptide having FDH activity is from the genus Candida sp., for example Candida boidinii, and can be, in some additional embodiments, the polypeptide having FDH activity having the amino acid sequence of SEQ ID NO: 5, being a variant of the amino acid sequence of SEQ ID NO: 5 or being a fragment of the amino acid sequence of SEQ ID NO: 5.

In an embodiment, the polypeptide having FDH activity is from the genus Lactobacillus sp., for example Lactobacillus buchneri, and can be, in some additional embodiments, the polypeptide having FDH activity having the amino acid sequence of SEQ ID NO: 21, 25 or 26, being a variant of the amino acid sequence of SEQ ID NO: 21, 25 or 26 or being a fragment of the amino acid sequence of SEQ ID NO: 21, 25 or 26. In some additional embodiments, the heterologous nucleic acid encoding the polypeptide having FDH activity can have the nucleic acid sequence of SEQ ID NO: 22. In embodiments in which the polypeptide has FDH activity having the amino acid sequence of SEQ ID NO: 21, 25 or 26, is a variant of the amino acid sequence of SEQ ID NO: 21, 25 or 26 or is a fragment of the amino acid sequence of SEQ ID NO: 21, 25 or 26, the recombinant yeast host cell expressing such polypeptide can include native FDH genes.

In an embodiment, the polypeptide having FDH activity is from the genus Granulicella sp., for example Granulicella mallensis, and can be, in some additional embodiments, the polypeptide having FDH activity having the amino acid sequence of SEQ ID NO: 23, being a variant of the amino acid sequence of SEQ ID NO: 23 or being a fragment of the amino acid sequence of SEQ ID NO: 23. In some additional embodiments, the heterologous nucleic acid encoding the polypeptide having FDH activity can have the nucleic acid sequence of SEQ ID NO: 24. In embodiments in which the polypeptide has FDH activity having the amino acid sequence of SEQ ID NO: 23, is a variant of the amino acid sequence of SEQ ID NO: 23 or is a fragment of the amino acid sequence of SEQ ID NO: 23, the recombinant yeast host cell expressing such polypeptide can include native FDH genes.

In an embodiment, the polypeptide having FDH activity is from the genus Bacillus sp., for example Bacillus stabilis, and can be, in some additional embodiments, the polypeptide having FDH activity having the amino acid sequence of SEQ ID NO: 27, being a variant of the amino acid sequence of SEQ ID NO: 27 or being a fragment of the amino acid sequence of SEQ ID NO: 27. In some additional embodiments, the heterologous nucleic acid encoding the polypeptide having FDH activity can have the nucleic acid sequence of SEQ ID NO: 28, 29 or 30. In embodiments in which the polypeptide has FDH activity having the amino acid sequence of SEQ ID NO: 27, is a variant of the amino acid sequence of SEQ ID NO: 27 or is a fragment of the amino acid sequence of SEQ ID NO: 27, the recombinant yeast host cell expressing such polypeptide can include native FDH genes.

The second or third heterologous nucleic acid molecules encoding an heterologous polypeptide having FDH activity can also include a signal sequence for targeting the expression of the polypeptide having FDH activity to the mitochondria. This signal sequence is referred to as a “mitochondrial targeting sequence” and is usually located upstream on the heterologous nucleic acid molecule and in frame with the coding sequence for the polypeptide having FDH activity. The mitochondrial targeting sequence can be cleaved, but not necessarily, from the polypeptide upon its translocation to the mitochondria. As such, the mitochondrial targeting sequence can be present or absent in the mature form of the polypeptide having FDH activity. The mitochondrial targeting sequence that can be used can be derived from any polypeptide expressed in the mitochondria that is expressed in eukaryotes. In some embodiments, the mitochondrial targeting sequence is derived from a yeast, for example from Saccharomyces cerevisiae. In yet another embodiment, the mitochondrial targeting sequence is derived from a polypeptide expressed in the mitochondria, including, but not limited to CYB2. In still a further embodiment, the mitochondrial targeting sequence has the amino acid sequence of SEQ ID NO: 11, is a variant of the amino acid sequence of SEQ ID NO: 11 (having the ability to target the expression of the polypeptide in the mitochondria) or is a fragment of the amino acid sequence of SEQ ID NO: 11 (having the ability to target the expression of the polypeptide in the mitochondria).

The second or third heterologous nucleic acid sequence can be present in one, two, three, four, five, six, seven, eighth, nine or ten or more copies in the recombinant and/or the further yeast host cell. In some embodiments, no more than ten, nine, eight, seven, six, five, four, three, two or a single copy of the second or third heterologous nucleic acid sequence is present in the recombinant and/or the further yeast host cell. In such embodiment, the second or third heterologous nucleic acid sequence can also include a constitutive promoter for expressing the polypeptide having FDH activity. Even in embodiments in which the second or third heterologous nucleic acid sequence is present in the recombinant or in the further yeast host cell, the present disclosure contemplates inactivating one or more native FDH genes in the recombinant or the further yeast host cell (e.g., including the fourth genetic modification in the recombinant or the further yeast host cell).

Additional Genetic Modifications

The recombinant yeast host cell of the present disclosure can also include one or more additional genetic modifications. These additional modifications can, for example, increase the fermentation abilities of the recombinant yeast host cell and, in some embodiments, increase ethanol yield and/or decrease glycerol yield of the recombinant yeast host cell during fermentation. In some embodiments, the recombinant yeast host cell can have a sixth genetic modification allowing or increasing the expression of an heterologous saccharolytic enzyme (when compared to a native yeast host cell lacking the sixth genetic modification), a seventh genetic modification allowing or increasing the utilization of acetyl-CoA (when compared to a native yeast host cell lacking the seventh genetic modification), an eighth genetic modification for reducing/limiting the production of glycerol (when compared to a native yeast host cell lacking the eighth genetic modification) and/or an ninth genetic modification for facilitating glycerol transport into the recombinant yeast host cell (when compared to a native yeast host cell lacking the ninth genetic modification). In an embodiment, the recombinant host cell has at least one of the sixth, seventh, eighth or ninth genetic modification. In another embodiment, the recombinant host cell has at least two of the sixth, seventh, eighth or ninth genetic modifications. In an embodiment, the recombinant host cell has at least three of the sixth, seventh, eighth or ninth genetic modifications. In an embodiment, the recombinant host cell has the sixth, seventh, eighth and ninth genetic modifications.

As indicated above, the recombinant yeast host cell can have a sixth genetic modification allowing the expression of an heterologous saccharolytic enzyme. As used in the context of the present disclosure, a “saccharolytic enzyme” can be any enzyme involved in carbohydrate digestion, metabolism and/or hydrolysis, including amylases, cellulases, hemicellulases, cellulolytic and amylolytic accessory enzymes, inulinases, levanases, and pentose sugar utilizing enzymes. amylolytic enzyme. In an embodiment, the saccharolytic enzyme is an amylolytic enzyme. As used herein, the expression “amylolytic enzyme” refers to a class of enzymes capable of hydrolyzing starch or hydrolyzed starch. Amylolytic enzymes include, but are not limited to alpha-amylases (EC 3.2.1.1, sometimes referred to fungal alpha-amylase, see below), maltogenic amylase (EC 3.2.1.133), glucoamylase (EC 3.2.1.3), glucan 1,4-alpha-maltotetraohydrolase (EC 3.2.1.60), pullulanase (EC 3.2.1.41), iso-amylase (EC 3.2.1.68) and amylomaltase (EC 2.4.1.25). In an embodiment, the one or more amylolytic enzymes can be an alpha-amylase from Aspergillus oryzae, a maltogenic alpha-amylase from Geobacillus stearothermophilus, a glucoamylase from Saccharomycopsis fibuligera, a glucan 1,4-alpha-maltotetraohydrolase from Pseudomonas saccharophila, a pullulanase from Bacillus naganoensis, a pullulanase from Bacillus acidopullulyticus, an iso-amylase from Pseudomonas amyloderamosa, and/or amylomaltase from Thermus thermophilus. Some amylolytic enzymes have been described in WO2018/167670 and are incorporated herein by reference.

In specific embodiments, the recombinant yeast host cell can bear one or more genetic modifications allowing for the production of an heterologous glucoamylase as the heterologous saccharolytic/amylolytic enzyme. Many microbes produce an amylase to degrade extracellular starches. In addition to cleaving the last α(1-4) glycosidic linkages at the non-reducing end of amylose and amylopectin, yielding glucose, γ-amylase will cleave α(1-6) glycosidic linkages. The heterologous glucoamylase can be derived from any organism. In an embodiment, the heterologous polypeptide is derived from a γ-amylase, such as, for example, the glucoamylase of Saccharomycoces filbuligera (e.g., encoded by the glu 0111 gene). Examples of recombinant yeast host cells bearing such first genetic modifications are described in WO 2011/153516 as well as in WO 2017/037614 and herewith incorporated in its entirety. In an embodiment, the sixth genetic modification comprises the introduction of an heterologous nucleic acid molecule encoding a polypeptide of SEQ ID NO: 9, a variant thereof or a fragment thereof. In some embodiments, the sixth genetic modification is encoded by a nucleic acid sequence of SEQ ID NO: 18 or 19, a variant of the nucleic acid sequence of SEQ ID NO: 18 or 19 or a fragment of the nucleic acid sequence of SEQ ID NO: 18 or 19.

Alternatively or in combination, the recombinant yeast host cell can bear one or more seventh genetic modifications for utilizing acetyl-CoA for example, by providing or increasing acetaldehyde and/or alcohol dehydrogenase activity. Acetyl-coA can be converted to an alcohol such as ethanol using first an acetaldehyde dehydrogenase and then an alcohol dehydrogenase. Acylating acetaldehyde dehydrogenases (E.C. 1.2.1.10) are known to catalyze the conversion of acetaldehyde into acetyl-coA in the presence of coA. Alcohol dehydrogenases (E.C. 1.1.1.1) are known to be able to catalyze the conversion of acetaldehyde into ethanol. The acetaldehyde dehydrogenase and alcohol dehydrogenase activity can be provided by a single polypeptide (e.g., a bifunctional acetaldehyde/alcohol dehydrogenase) or by a combination of more than one polypeptide (e.g., an acetaldehyde dehydrogenase and an alcohol dehydrogenase). In embodiments in which the acetaldehyde/alcohol dehydrogenase activity is provided by more than one polypeptide, it may not be necessary to provide the combination of polypeptides in a recombinant form in the recombinant yeast host cell as the cell may have some pre-existing acetaldehyde or alcohol dehydrogenase activity. In such embodiments, the seventh genetic modification can include providing one or more heterologous nucleic acid molecule encoding one or more of an heterologous acetaldehyde dehydrogenase (AADH), an heterologous alcohol dehydrogenase (ADH) and/or heterologous bifunctional acetylaldehyde/alcohol dehydrogenases (ADHE). For example, the seventh genetic modification can comprise introducing an heterologous nucleic acid molecule encoding an acetaldehyde dehydrogenase. In another example, the seventh genetic modification can comprise introducing an heterologous nucleic acid molecule encoding an alcohol dehydrogenase. In still another example, the seventh genetic modification can comprise introducing at least two heterologous nucleic acid molecules, a first one encoding an heterologous acetaldehyde dehydrogenase and a second one encoding an heterologous alcohol dehydrogenase. In another embodiment, the seventh genetic modification comprises introducing an heterologous nucleic acid encoding an heterologous bifunctional acetylaldehyde/alcohol dehydrogenases (AADH) such as those described in U.S. Pat. No. 8,956,851 and WO 2015/023989. Heterologous AADHs of the present disclosure include, but are not limited to, the ADHE polypeptides or a polypeptide encoded by an adhe gene ortholog. In an embodiment, the AADH has the amino acid sequence of SEQ ID NO: 12, is a variant of the amino acid sequence of SEQ ID NO: 12 or is a fragment of the amino acid sequence of SEQ ID NO: 12. In such embodiment, the seventh genetic modification can comprise introducing an heterologous nucleic acid molecule encoding a polypeptide having the amino acid sequence of SEQ ID NO: 12, being a variant of the amino acid sequence of SEQ ID NO: 12 or being a fragment of the amino acid sequence of SEQ ID NO: 12. The seventh genetic modification can comprising introducing an heterologous nucleic acid molecule having the nucleic acid sequence of SEQ ID NO: 14 or 15, being a variant of a nucleic acid sequence of SEQ ID NO: 14 or 15 or being a fragment of a nucleic acid sequence of SEQ ID NO: 14 or 15.

Alternatively or in combination, the recombinant yeast host cell can also include one or more eighth genetic modifications limiting the production of glycerol. For example, the eighth genetic modification can be a genetic modification leading to the reduction in the production, and in an embodiment to the inhibition in the production, of one or more native enzymes that function to produce glycerol. As used in the context of the present disclosure, the expression “reducing the production of one or more native enzymes that function to produce glycerol” refers to a genetic modification which limits or impedes the expression of genes associated with one or more native polypeptides (in some embodiments enzymes) that function to produce glycerol, when compared to a corresponding yeast strain which does not bear such genetic modification. In some instances, the additional genetic modification reduces but still allows the production of one or more native polypeptides that function to produce glycerol. In other instances, the genetic modification inhibits the production of one or more native enzymes that function to produce glycerol. Polypeptides that function to produce glycerol refer to polypeptides which are endogenously found in the recombinant yeast host cell. Native enzymes that function to produce glycerol include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide) or the gpp2 gene (encoding the GPP2 polypeptide). Examples of recombinant yeast host cells bearing such genetic modification(s) leading to the reduction in the production of one or more native enzymes that function to produce glycerol are described in WO 2012/138942. In some embodiments, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene and the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). In still another embodiment (in combination or alternative to the genetic modification described above).

In yet another embodiment, the recombinant yeast host cell does not bear an eighth genetic modification and includes its native genes coding for the GPP/GDP polypeptides. As such, in some embodiments, there are no genetic modifications leading to the reduction in the production of one or more native enzymes that function to produce glycerol in the recombinant yeast host cell.

As used in the context of the present disclosure, the expression “native polypeptides that function to produce glycerol” refers to polypeptides which are endogenously found in the recombinant yeast host cell. Native enzymes that function to produce glycerol may include, but are not limited to, the GPD1 and the GPD2 polypeptide (also referred to as GPD1 and GPD2 respectively) as well as the GPP1 and the GPP2 polypeptides (also referred to as GPP1 and GPP2 respectively). In an embodiment, the recombinant yeast host cell bears a genetic modification in at least one of the gpd1 gene (encoding the GPD1 polypeptide), the gpd2 gene (encoding the GPD2 polypeptide), the gpp1 gene (encoding the GPP1 polypeptide), the gpp2 gene (encoding the GPP2 polypeptide), orthologs thereof or paralogs thereof. In another embodiment, the recombinant yeast host cell bears a genetic modification in at least two of the gpd1 gene (encoding the GPD1 polypeptide), the gpd2 gene (encoding the GPD2 polypeptide), the gpp1 gene (encoding the GPP1 polypeptide), the gpp2 gene (encoding the GPP2 polypeptide), orthologs thereof or paralogs thereof. In still another embodiment, the recombinant yeast host cell bears a genetic modification in each of the gpd1 gene (encoding the GPD1 polypeptide), the gpd2 gene (encoding the GPD2 polypeptide), orthologs thereof and paralogs thereof. Examples of recombinant yeast host cells bearing such genetic modification(s) leading to the reduction in the production of one or more native enzymes that function to produce glycerol or regulating glycerol synthesis are described in WO 2012/138942. Preferably, the recombinant yeast host cell has a genetic modification (such as a genetic deletion or insertion) only in one enzyme that functions to produce glycerol, in the gpd2 gene, which would cause the host cell to have a knocked-out gpd2 gene. In some embodiments, the recombinant yeast host cell can have a genetic modification in the gpd1 gene, the gpd2 gene resulting is a recombinant yeast host cell being knock-out for the gpd1 gene and the gpd2 gene. In some specific embodiments, the recombinant yeast host cell can have be a knock-out for the gpd1 gene and have duplicate copies of the gpd2 gene (in some embodiments, under the control of the gpd1 promoter). Alternatively, the recombinant yeast host cell of the present disclosure can also bear and express its native polypeptides that function to produce glycerol. In such embodiment, the recombinant yeast host cell can retain its native gpd1, gpd2, gpp1 and gpp2 genes in an unaltered (e.g., wild-type) form.

Alternatively or in combination, the recombinant yeast host cell can also include one or more ninth genetic modifications facilitating the transport of glycerol in the recombinant yeast host cell. For example, the ninth genetic modification can be a genetic modification leading to the increase in activity of one or more native enzymes that function to transport glycerol. Native enzymes that function to transport glycerol synthesis include, but are not limited to, the FPS1 polypeptide as well as the STL1 polypeptide. The FPS1 polypeptide is a glycerol exporter and the STL1 polypeptide functions to import glycerol in the recombinant yeast host cell. By either reducing or inhibiting the expression of the FPS1 polypeptide and/or increasing the expression of the STL1 polypeptide, it is possible to control, to some extent, glycerol transport.

The STL1 polypeptide is natively expressed in yeasts and fungi, therefore the heterologous polypeptide functioning to import glycerol can be derived from yeasts and fungi. STL1 genes encoding the STL1 polypeptide include, but are not limited to, Saccharomyces cerevisiae Gene ID: 852149, Candida albicans, Kluyveromyces lactis Gene ID: 2896463, Ashbya gossypii Gene ID: 4620396, Eremothecium sinecaudum Gene ID: 28724161, Torulaspora delbrueckii Gene ID: 11505245, Lachancea thermotolerans Gene ID: 8290820, Phialophora attae Gene ID: 28742143, Penicillium digitatum Gene ID: 26229435, Aspergillus oryzae Gene ID: 5997623, Aspergillus fumigatus Gene ID: 3504696, Talaromyces atroroseus Gene ID: 31007540, Rasamsonia emersonii Gene ID: 25315795, Aspergillus flavus Gene ID: 7910112, Aspergillus terreus Gene ID: 4322759, Penicillium chrysogenum Gene ID: 8310605, Alternaria alternata Gene ID: 29120952, Paraphaeosphaeria sporulosa Gene ID: 28767590, Pyrenophora tritici-repentis Gene ID: 6350281, Metarhizium robertsii Gene ID: 19259252, Isaria fumosorosea Gene ID: 30023973, Cordyceps militaris Gene ID: 18171218, Pochonia chlamydosporia Gene ID: 28856912, Metarhizium majus Gene ID: 26274087, Neofusicoccum parvum Gene ID: 19029314, Diplodia corticola Gene ID: 31017281, Verticillium dahliae Gene ID: 20711921, Colletotrichum gloeosporioides Gene ID: 18740172, Verticillium albo-atrum Gene ID: 9537052, Paracoccidioides lutzii Gene ID: 9094964, Trichophyton rubrum Gene ID: 10373998, Nannizzia gypsea Gene ID: 10032882, Trichophyton verrucosum Gene ID: 9577427, Arthroderma benhamiae Gene ID: 9523991, Magnaporthe oryzae Gene ID: 2678012, Gaeumannomyces graminis var. tritici Gene ID: 20349750, Togninia minima Gene ID: 19329524, Eutypa lata Gene ID: 19232829, Scedosporium apiospermum Gene ID: 27721841, Aureobasidium namibiae Gene ID: 25414329, Sphaerulina musiva Gene ID: 27905328 as well as Pachysolen tannophilus GenBank Accession Numbers JQ481633 and JQ481634, Saccharomyces paradoxus STL1 and Pichia sorbitophilia. In an embodiment, the STL1 polypeptide is encoded by Saccharomyces cerevisiae Gene ID: 852149. In still another embodiment, the STL1 polypeptide can have the amino acid of SEQ ID NO: 10, be a variant of the amino acid of SEQ ID NO: 10 or be a fragment of the amino acid of SEQ ID NO: 10. In another embodiment, the recombinant yeast host cell comprises an heterologous nucleic acid sequence having the nucleic acid sequence of SEQ ID NO: 20.

Combinations

The recombinant yeast host cell described herein can be provided as a combination with the further yeast cell described herein. In such combination, the recombinant yeast host cell can be provided in a distinct container from the further yeast host cell. The recombinant and further yeast host cell can be provided as a cell concentrate. The cell concentrate comprising the recombinant and/or further yeast host cell can be obtained, for example, by propagating the yeast host cells in a culture medium and removing at least one components of the medium comprising the propagated yeast host cell. This can be done, for example, by dehydrating, filtering (including ultra-filtrating) and/or centrifuging the medium comprising the propagated yeast host cell. In an embodiment, the recombinant and/or the further yeast host cell is provided as cream in the combination.

The present disclosure also provides for fermenting the biomass in the presence of the recombinant yeast host cell and the further yeast host cell. In the process described herein, the recombinant yeast host cell can be added to the biomass prior to the further yeast host cell. Alternatively, the further yeast host cell can be added to the biomass prior to the recombinant yeast host cell. Also, the recombinant yeast host cell and the further yeast host cell can be added at the same time to the biomass.

Process for Converting Biomass

The recombinant yeast host cells (or combinations comprising same) described herein can be used to improve fermentation yield while maintaining yeast robustness during fermentation especially in the presence of a stressor such as, for example, lactic acid, formic acid and/or a bacterial contamination (that can be associated, in some embodiments, the an increase in lactic acid during fermentation), an increase in pH, a reduction in aeration, elevated temperatures or combinations. The fermented product can be an alcohol, such as, for example, ethanol, isopropanol, n-propanol, 1-butanol, methanol, acetone and/or 1, 2 propanediol. In an embodiment, the fermented product is ethanol. The fermented product can be, for example, an heterologous polypeptide that is expressed in a recombinant fashion by the recombinant yeast host cell.

The biomass that can be fermented with the recombinant yeast host cells or co-cultures with a further yeast cell as described herein includes any type of biomass known in the art and described herein. For example, the biomass can include, but is not limited to, starch, sugar and lignocellulosic materials. Starch materials can include, but are not limited to, mashes such as corn, wheat, rye, barley, rice, or milo. Sugar materials can include, but are not limited to, sugar beets, artichoke tubers, sweet sorghum, molasses or cane. The terms “lignocellulosic material”, “lignocellulosic substrate” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants and sugar-processing residues. The terms “hemicellulosics”, “hemicellulosic portions” and “hemicellulosic fractions” mean the non-lignin, non-cellulose elements of lignocellulosic material, such as but not limited to hemicellulose (i.e., comprising xyloglucan, xylan, glucuronoxylan, arabinoxylan, mannan, glucomannan and galactoglucomannan), pectins (e.g., homogalacturonans, rhamnogalacturonan I and II, and xylogalacturonan) and proteoglycans (e.g., arabinogalactan-polypeptide, extensin, and pro line-rich polypeptides).

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; succulents, such as but not limited to, agave; and forestry wastes, such as but not limited to, recycled wood pulp fiber, sawdust, hardwood (e.g., poplar, oak, maple, birch, willow), softwood, or any combination thereof. Lignocellulosic material may comprise one species of fiber; alternatively, lignocellulosic material may comprise a mixture of fibers that originate from different lignocellulosic materials. Other lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Substrates for cellulose activity assays can be divided into two categories, soluble and insoluble, based on their solubility in water. Soluble substrates include cellodextrins or derivatives, carboxymethyl cellulose (CMC), or hydroxyethyl cellulose (HEC). Insoluble substrates include crystalline cellulose, microcrystalline cellulose (Avicel), amorphous cellulose, such as phosphoric acid swollen cellulose (PASC), dyed or fluorescent cellulose, and pretreated lignocellulosic biomass. These substrates are generally highly ordered cellulosic material and thus only sparingly soluble.

It will be appreciated that suitable lignocellulosic material may be any feedstock that contains soluble and/or insoluble cellulose, where the insoluble cellulose may be in a crystalline or non-crystalline form. In various embodiments, the lignocellulosic biomass comprises, for example, wood, corn, corn stover, sawdust, bark, molasses, sugarcane, leaves, agricultural and forestry residues, grasses such as switchgrass, ruminant digestion products, municipal wastes, paper mill effluent, newspaper, cardboard or combinations thereof.

Paper sludge is also a viable feedstock for lactate or acetate production. Paper sludge is solid residue arising from pulping and paper-making, and is typically removed from process wastewater in a primary clarifier. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

The process of the present disclosure contacting the recombinant host cells described herein with a biomass so as to allow the conversion of at least a part of the biomass into the fermentation product (e.g., an alcohol such as ethanol). In an embodiment, the biomass or substrate to be hydrolyzed is a lignocellulosic biomass and, in some embodiments, it comprises starch (in a gelatinized or raw form). The process can include, in some embodiments, heating the lignocellulosic biomass prior to fermentation to provide starch in a gelatinized form.

The fermentation process can be performed at temperatures of at least about 20° C., about 21° C., about 22° C., about 23° C., about 24° C., about 25° C., about 26° C., about 27° C., about 28° C., about 29° C., about 30° C., about 31° C., about 32° C., about 330, about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the production of ethanol from cellulose can be performed, for example, at temperatures above about 30° C., about 31° C., about 32° C., about 33° C., about 34° C., about 35° C., about 36° C., about 37° C., about 38° C., about 39° C., about 40° C., about 41° C., about 42° C., or about 43° C., or about 44° C., or about 45° C., or about 50° C. In some embodiments, the recombinant microbial host cell can produce ethanol from cellulose at temperatures from about 30° C. to 60° C., about 30° C. to 55° C., about 30° C. to 50° C., about 40° C. to 60° C., about 40° C. to 55° C. or about 40° C. to 50° C.

In some embodiments, the process can be used to produce ethanol at a particular rate. For example, in some embodiments, ethanol is produced at a rate of at least about 0.1 mg per hour per liter, at least about 0.25 mg per hour per liter, at least about 0.5 mg per hour per liter, at least about 0.75 mg per hour per liter, at least about 1.0 mg per hour per liter, at least about 2.0 mg per hour per liter, at least about 5.0 mg per hour per liter, at least about 10 mg per hour per liter, at least about 15 mg per hour per liter, at least about 20.0 mg per hour per liter, at least about 25 mg per hour per liter, at least about 30 mg per hour per liter, at least about 50 mg per hour per liter, at least about 100 mg per hour per liter, at least about 200 mg per hour per liter, at least about 300 mg per hour per liter, at least about 400 mg per hour per liter, at least about 500 mg per hour per liter, at least about 600 mg per hour per liter, at least about 700 mg per hour per liter, at least about 800 mg per hour per liter, at least about 900 mg per hour per liter, at least about 1 g per hour per liter, at least about 1.5 g per hour per liter, at least about 2 g per hour per liter, at least about 2.5 g per hour per liter, at least about 3 g per hour per liter, at least about 3.5 g per hour per liter, at least about 4 g per hour per liter, at least about 4.5 g per hour per liter, at least about 5 g per hour per liter, at least about 5.5 g per hour per liter, at least about 6 g per hour per liter, at least about 6.5 g per hour per liter, at least about 7 g per hour per liter, at least about 7.5 g per hour per liter, at least about 8 g per hour per liter, at least about 8.5 g per hour per liter, at least about 9 g per hour per liter, at least about 9.5 g per hour per liter, at least about 10 g per hour per liter, at least about 10.5 g per hour per liter, at least about 11 g per hour per liter, at least about 11.5 g per hour per liter, at least about 12 g per hour per liter, at least about 12.5 g per hour per liter, at least about 13 g per hour per liter, at least about 13.5 g per hour per liter, at least about 14 g per hour per liter, at least about 14.5 g per hour per liter or at least about 15 g per hour per liter.

Ethanol production can be measured using any method known in the art. For example, the quantity of ethanol in fermentation samples can be assessed using HPLC analysis. Many ethanol assay kits are commercially available that use, for example, alcohol oxidase enzyme based assays.

The present invention will be more readily understood by referring to the following examples which are given to illustrate the invention rather than to limit its scope.

### Example I—Modulation of FDH Activity During Fermentation

Tables 9 below summarizes the genotype of the various Saccharomyces cerevisiae strains used in this example.

TABLE 9A
Genotype information of the Saccharomyces cerevisiae
strains used in this example.

| | | Inactivated |
|---|---|---|
| Strain | Genes overexpressed | genes |
| M2390 (wild-type) | None | None |
| M8841 | ADHE | fdh1Δ |
| | PFLA (4 copies) | fdh2Δ |
| | PFLB (4 copies) | gpd2Δ |
| | GLU | fcy1Δ |
| M12156 | ADHE | fdh1Δ |
| | PFLA (4 copies) | fdh2Δ |
| | PFLB (4 copies) | gpd2Δ |
| | GLU | fcy1Δ |
| | STL1 | |
| M15052 | Same as M12156 | Same as M12156 |
| | 4X FDH1 | |
| M15418 | Same as M12156 | Same as M12156 |
| | 2X FDH1-CYB2 | |
| M15419 | Same as M12156 | Same as M12156 |
| | 2X FDH1 | |
| M15425 | Same as M12156 | Same as M12156 |
| | 2X FDH3 | |
| M15427 | Same as M12156 | Same as M12156 |
| | 2X FDH1-QRN | |
| M15430 | Same as M12156 | Same as M12156 |
| | 2X FDH3-CYB2 | |
| M17952 | ADHE | fcy1Δ |
| | PFLA | ylr296WΔ |
| | PFLB | |
| | GLU | |
| | STL1 | |
| | FDH1 | |
| M8279 | None | None |
| M18971 | ADHE | ime1Δ |
| | PFLA | |
| | PFLB | |
| M20345 | ADHE | ime1Δ |
| | PFLA | ylr296WΔ |
| | PFLB | |
| | MP1180 (SEQ ID NO: 21) | |
| | expressed under the control | |
| | of the adh1p promoter | |
| M20341 | ADHE | ime1Δ |
| | PFLA | ylr296WΔ |
| | PFLB | |
| | MP1180 (SEQ ID NO: 21) | |
| | expressed under the control | |
| | of the tef2p promoter | |
| M20344 | ADHE | ime1Δ |
| | PFLA | ylr296WΔ |
| | PFLB | |
| | MP1180 (SEQ ID NO: 21) | |
| | expressed under the control | |
| | of the ssa1p promoter | |
| M20999 | ADHE | ime1Δ |
| | PFLA | ylr296WΔ |
| | PFLB | |
| | FDH1 expressed under the | |
| | control of the tef2p promoter | |
| M21000 | ADHE | ime1Δ |
| | PFLA | ylr296WΔ |
| | PFLB | |
| | FDH1 expressed under the | |
| | control of the adh1p promoter | |
| M21001 | ADHE | ime1Δ |
| | PFLA | ylr296WΔ |
| | PFLB | |
| | FDH1 expressed under the | |
| | control of the ssa1p promoter | |
| M23016 | ADHE | ime1Δ |
| | PFLA | |
| | PFLB | |
| | G199A (SEQ ID NO: 25) under | |
| | the control of the tef2 promoter | |
| M23017 | ADHE | ime1Δ |
| | PFLA | |
| | PFLB | |
| | Q222A (SEQ ID NO: 26) under | |
| | the control of the tef2 promoter | |

The following abbreviations are used: ADHE refers to an alcohol dehydrogenase having the amino acid sequence of SEQ ID NO: 8, PFLA refers to a pyruvate formate lyase having the amino acid sequence of SEQ ID NO: 6, PFLB refers to a pyruvate formate lyase having the amino acid sequence of SEQ ID NO: 7, GLU refers to a glucoamylase having the amino acid sequence of SEQ ID NO: 9, STL1 refers to a glycerol transporter having the amino acid sequence of SEQ ID NO: 11, FDH1 refers to a formate dehydrogenase having the amino acid sequence of SEQ ID NO: 1, FDH1-QRN refers to a formate dehydrogenase having the amino acid sequence of SEQ ID NO: 2 and FDH3 refers to a formate dehydrogenase having the amino acid sequence of SEQ ID NO: 5. The expression “-CYB2” refers to the addition, at the N-terminal of the polypeptide of a mitochondrial targeting signal sequence (as described in Hou et al., 2010).

TABLE 9B
Genotype information of the Saccharomyces cerevisiae strains
used in the promoter screen of this example. All of the strains
are derived from M18971 and express ADHE (SEQ ID NO: 8), PFLA
(SEQ ID NO: 6), PFLB (SEQ ID NO: 7) and MP1180 (SEQ ID NO: 21).
The table provides the promoters used to express MP1180.

| | Promoter used to control the |
|---|---|
| Strain | expression of MP1180 |
| M23002 | tef2 promoter (tef2p) |
| M23003 | ssa1 promoter (ssa1p) |
| M23004 | adh1 promoter (adh1p) |
| M23005 | cdc19 promoter (cdc19p) |
| M23006 | tpi1 promoter (tpi1p) |
| M23007 | cyc1 promoter (cyc1p) |
| M23008 | pgk1 promoter (pgk1p) |
| M23009 | tdh2 promoter (tdh2p) |
| M23010 | eno2 promoter (eno2p) |
| M23011 | htx3 promoter (hxt3p) |
| M23012 | qcr8 promoter (qcr8p) |
| M23013 | tdh1 promoter (tdh1p) |
| M23014 | tdh3 promoter (tdh3p) |
| M23015 | hor7 promoter (hor7p) |

Strain propagation. Yeast strains were patched to agar plates containing 1% yeast extract, 2% peptone, 4% glucose and 2% agar (YPD₄₀) from glycerol stocks and were incubated overnight at 35° C. The following day, a loop of cells was inoculated into 30 mL of YPD₄₀ media and grown overnight at 35° C. The overnight cultures were added into the fermentation at a concentration of 0.3 g/L of dry cell weight (DCW).

Fermentation. YPD cultures (25 to 50 g) were inoculated into 30-32.5% total solids (TS) corn mash containing lactrol (7 mg/kg) and penicillin (9 mg/kg) in anaerobic vented serum bottles. For permissive fermentation, the recommended concentration of urea was added (165-700 ppm) as the concentration of urea is mash dependent. In some experiments, no urea was added for the stress conditions (lactic, or lactic/formic, bacteria/formic). Exogenous glucoamylase was added at 100%=0.6 A GU/gTS. The various strains were dosed at 50%-65%. For permissive fermentation, the strains were incubated at 33° C. for either 18 h or 48 h, followed by 31° C. for the remainder of the fermentation (150 rpm shaking). For the lactic stress fermentation, the vessels were incubated at 34° C. throughout or at 36° C. for the high temperature stress fermentation. For the lactic stress fermentation, 0.38% w/v lactic acid was added at T=18 h. In experiments containing formic stress, 0.4 g/L exogenous formate (in the form of sodium formate) was added. For the bacterial stress, rehydrated L. plantarum was added at a concentration of 6×10⁸ cells/mL at the beginning of fermentation. Endpoint samples were collected at 48 h-65 h and assayed by HPLC for metabolites. When cocultures were performed, the strains were combined at the ratio provided prior to the fermentation.

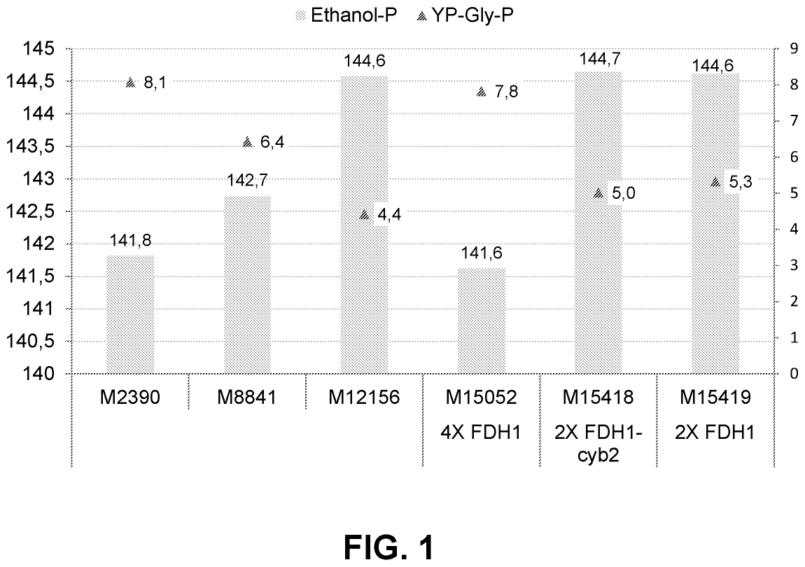

It is known that, in order to limit glycerol production and favor ethanol production, the synthesis of NADH can be limited by inactivating the native formate dehydrogenase in strains which also produce formate in recombinant yeast host cells (see FIG. 1 of WO2012138942).

FDH assay. Cells were grown in 5 mL of YPD overnight at 35° C. with agitation. Cultures were washed twice with ice-cold water and 1 mL of lysis buffer was added (Y-PER, 100 mM dithiothreitol, 1:1000 dilution mammalian protease inhibitor cocktail). The cells were incubated for 2 h at room temperature with shaking. The cells were pelleted and supernatant kept on ice for use in the assay. Three two-fold serial dilutions of the lysate were made and 50 μl transferred to PCR plate. Next, a buffer solution (10 mM potassium phosphate combined with 500 mM sodium formate, pH 7.5 final concentration) and a cofactor solution (NAD+ or NADP+, 10 mM diluted in water final concentration) were added to the cell lysates. Absorbance was determined at 340 nm every 30 seconds for 30 to 45 minutes. For the promoter library screen, cultures were grown anaerobically in 20 mL of YPD media. Cells were harvested and washed twice with ddH₂O. The cells were resuspended in 1 mL of ice-cold lysis buffer (10 mM triethanolamine pH 7, 2 mM MgCl₂, 1 mM dithiothreitol (DTT)). The cell suspension was disrupted via bead-beating using Zymo BashingBeah 0.5 mm tubes for 3×20 sec 4.0 m/s in a MP Fast-Prep homogenizer, cooling on ice in between cycles. Cells were pelleted and lysate filtered with 0.2 μm spin filter. Lysates were then used as described above the for FDH activity assays. BCA assay was used to determine total protein concentration in the cell lysate.

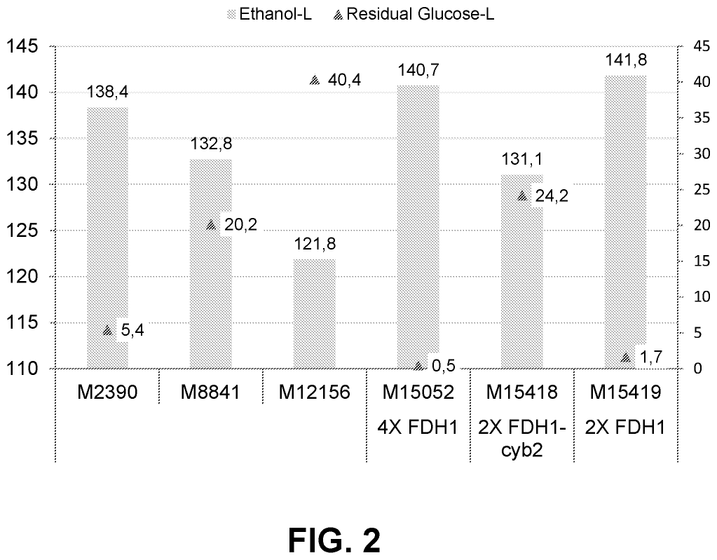

It was first determined if the deletion of the native formate dehydrogenase genes present in the strains had an impact on the fermentation yield in permissive and lactic stress conditions. As shown in FIGS. 1, 2 and 8, strains having native formate dehydrogenase genes (M2390, M8841 and M17952) showed a limited decrease in ethanol yield during lactic stress fermentation when compared to results obtained during permissive fermentation. Strain M12156, which includes a deletion in both of its native formate dehydrogenase genes, showed a more profound reduction in ethanolic yield and glucose consumption. Without wishing to be bound to theory, it is assumed that the accumulation of formate in strain M12156 may be detrimental to its robustness when submitted to a stressor, such as lactic acid. Interestingly, when strain M12156 was further modified to overexpress of 2 (M15419) or 4 (M15052) copies of S. cerevisiae's FDH1 gene, an increase in ethanolic yield was observed during lactic stress fermentation. In addition, when strain M12156 was modified to express S. cerevisiae's FDH1 inside the mitochondria (M15418), an increase in ethanolic yield was also observed during lactic stress fermentation.

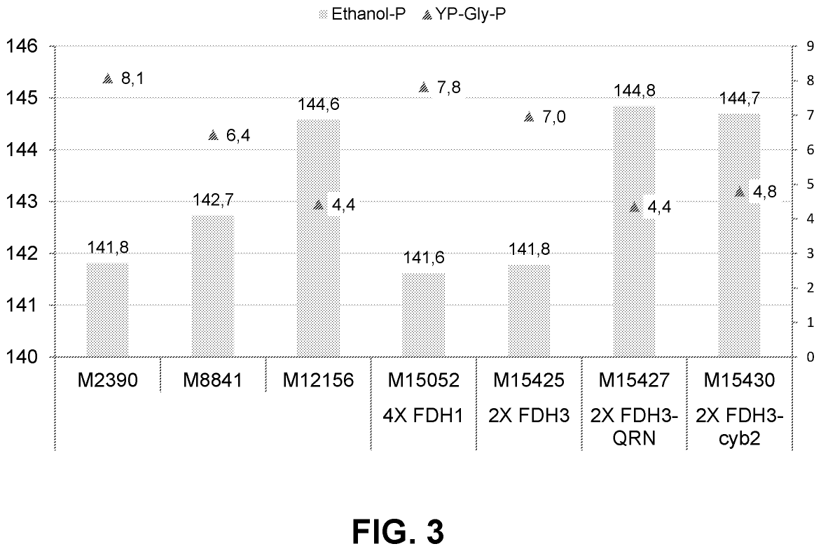

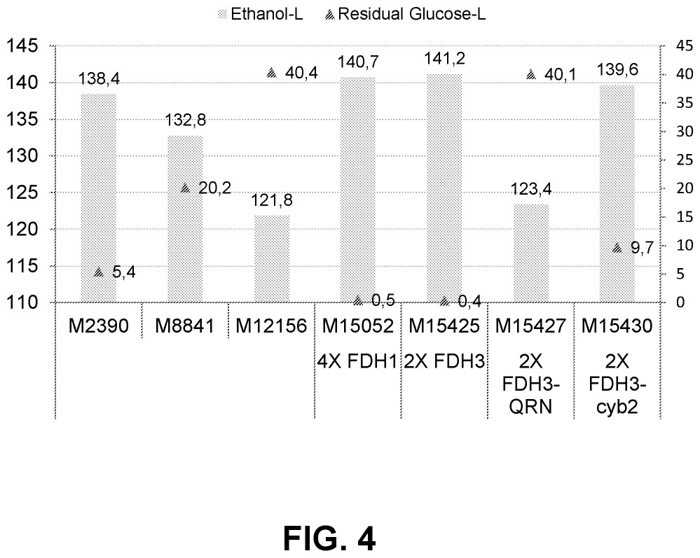

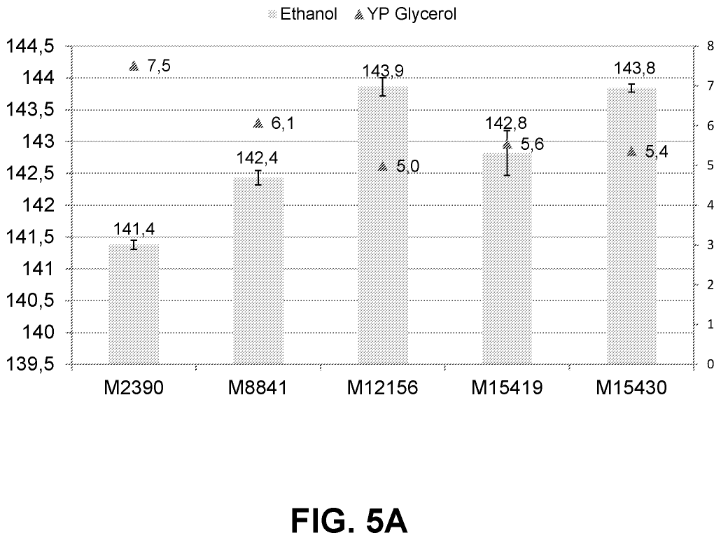

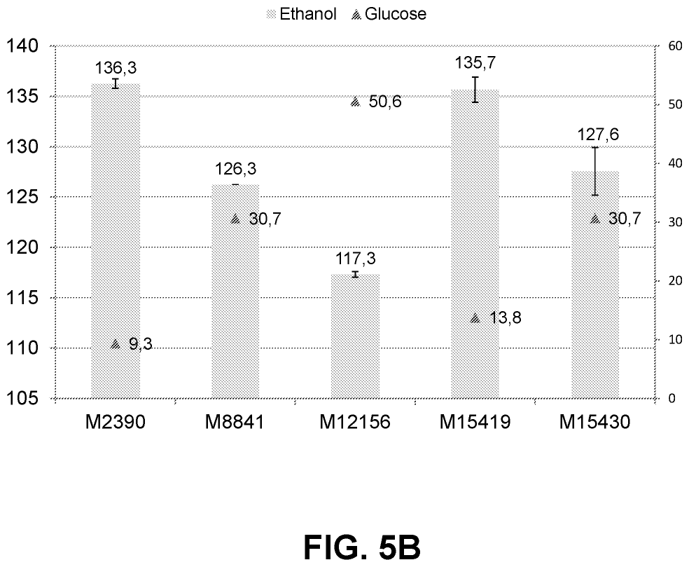

In order to determine if the effects observed were limited to a specific type of formate dehydrogenase, an heterologous formate dehydrogenase from Candida boidinii (FDH3) was introduced in strain M12156. Three different versions of FDH3 were expressed in M12156, the native FDH3 from C. boidinii (expressed in M15425), a mutated FDH3 which is known to exhibit specificity toward NADP⁺ instead of NAD⁺ (variant QRN expressed in M15427) or a FDH3 designed to be expressed in the mitochondria (by using the CYB2 mitochondrial signal sequence, expressed in M15430). As shown in FIGS. 3, 4 and 5, the introduction of FDH3 in all of its versions increased ethanolic yield and glucose consumption when compared to the results obtained with M12156 during lactic stress fermentation.

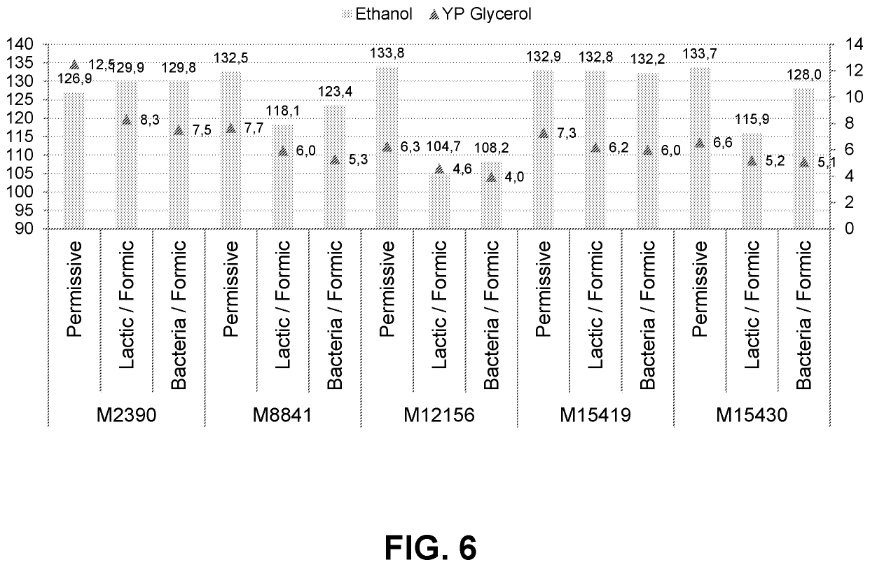

In order to determine if the expression of formate dehydrogenase can be advantageous to increase ethanolic yield in the presence of different types of stressors, fermentations were conducted in the presence of a combination stressors (e.g., lactic and formic acids (lactic/formic) or of bacteria and formic acid (bacteria/formic)). As shown on FIG. 6, in the presence of a combinations of stressors, strains M8841 and M12156 exhibited reduced ethanolic yield. The expression of FDH1 (M15419) or FDH3 (M15430) in a M12156 background increased in the ethanolic yields in stressful fermentations (when compared to M12156 without these additional modifications).

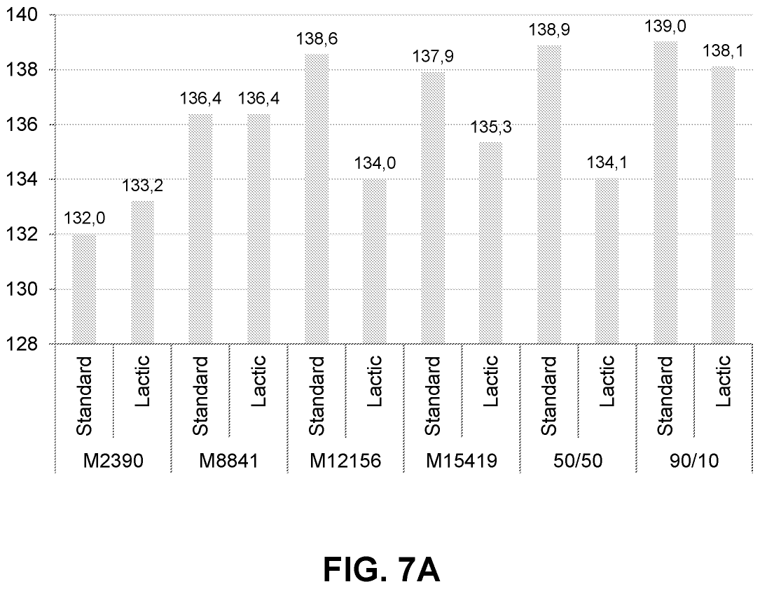

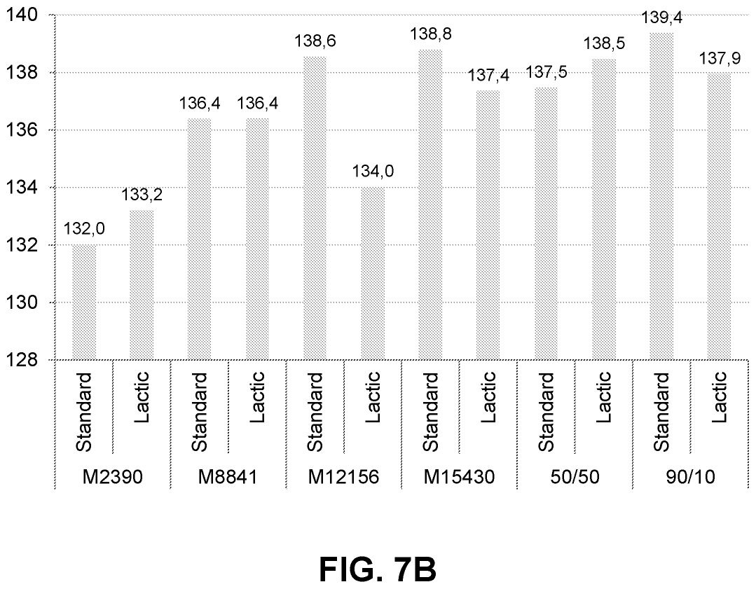

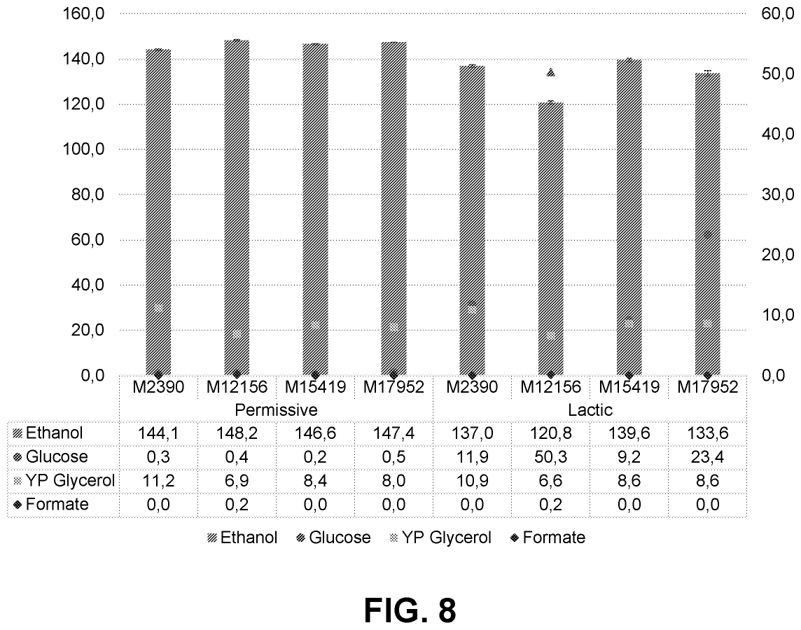

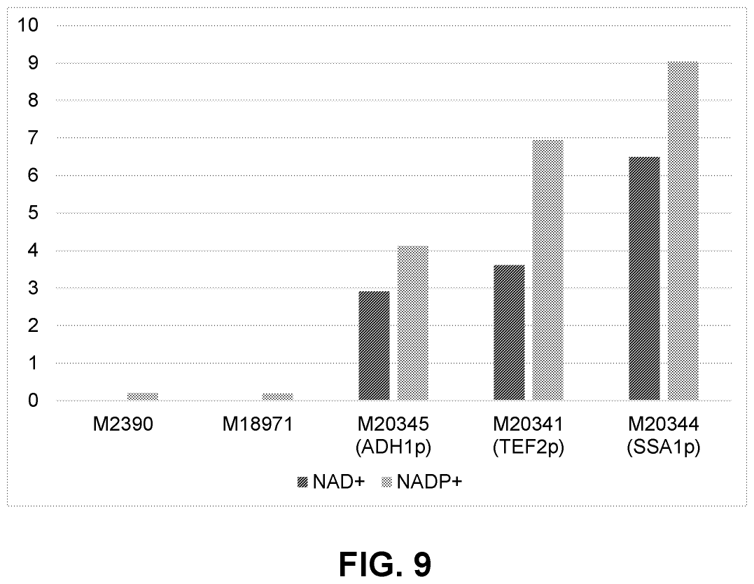

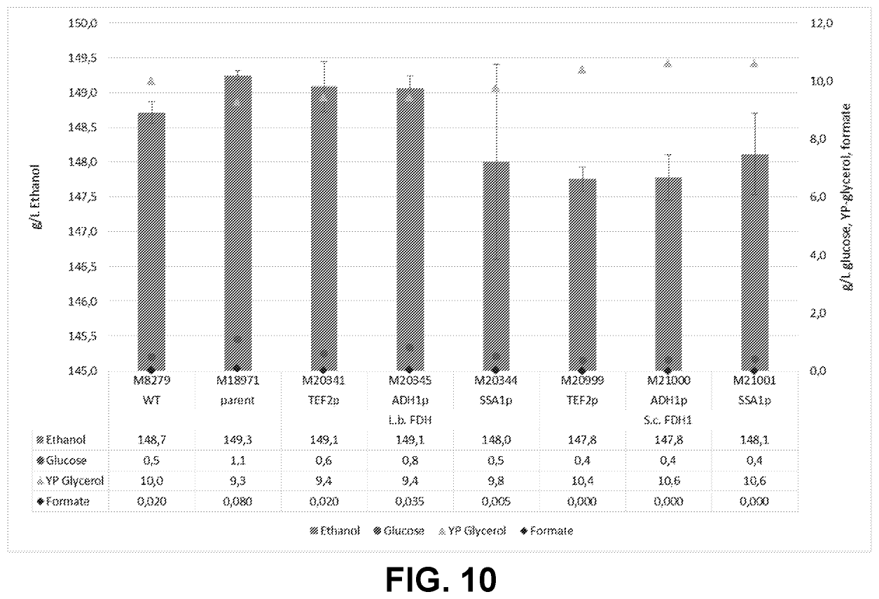

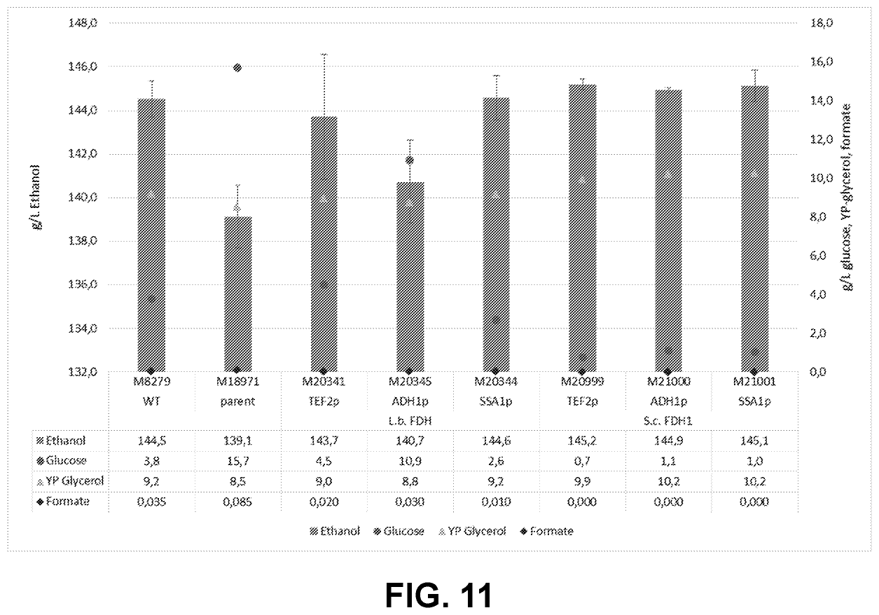

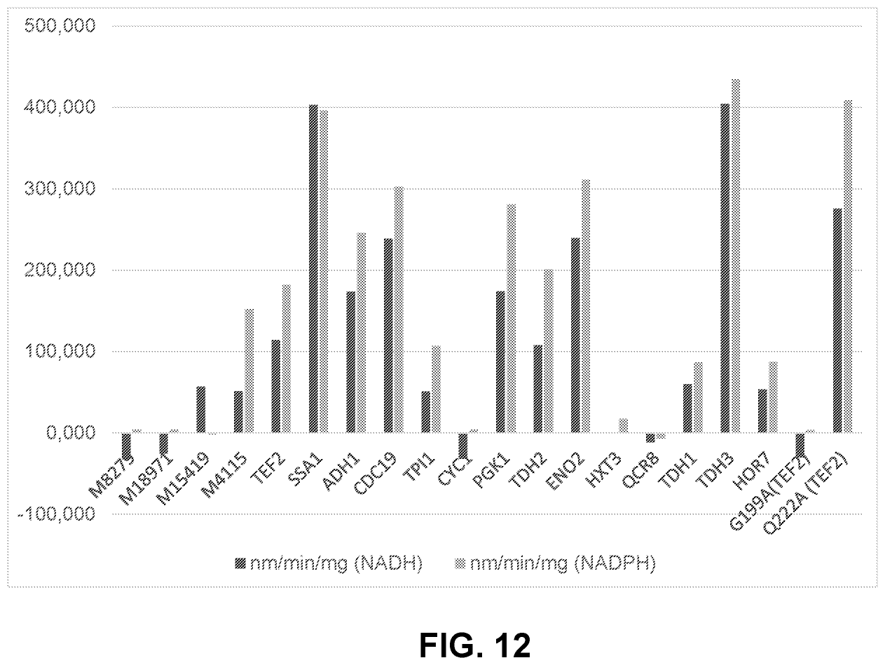

It was further determined if culturing a strain overexpressing a formate dehydrogenase polypeptide could restore the ethanolic yield during stress fermentation of another strain in which the endogenous formate dehydrogenase genes have been inactivated. In order to do so, strains M15419 and M15430, both expressing FDH1, have been blended with strain M12156 (in which the endogenous formate dehydrogenase genes have been inactivated). As shown in FIG. 7, the combination of strains overexpressing FDH1 with strain M12156 increased ethanol production, especially during lactic stress fermentation.

Additional strains were derived from strain M18971 which includes its native FDH genes. As shown on FIG. 9, the expression of native FDH genes (in strains M2390 and M18971) show little to no NAD+ or NADP+-formate dehydrogenase activity. However, strains expressing an heterologous NADP+-formate dehydrogenase from Lactobacillus buchneri (MP1180) exhibited higher NAD+ and NADP+-formate dehydrogenase activity than their parental counterpart (M18971). The strains expressing an heterologous NADP+-formate dehydrogenase from Lactobacillus buchneri (MP1180) exhibited higher NADP+ than NAD+-formate dehydrogenase activity.

The performance of the strains derived from strain M18971 was then determined in both a permissive and a stress (lactic acid) fermentation. When the results of FIGS. 10 and 11 are compared, it is observed that in the presence of a stressor, strains including native FDH genes and expressing an heterologous FDH polypeptide have an increase in ethanol yield when compared to the parental strain (M18971).

The heterologous NADP+-formate dehydrogenase from Lactobacillus buchneri (MP1180) was expressed under the control of different promoters (see Table 9B for a description of the different strains tested) and their resulting NAD+ and NADP+ activity was compared to control yeast strains (see Table 9A for a description of the different strains tested). The results of this promoter screen is shown in FIG. 12.

Mutated heterologous NADP+-formate dehydrogenase from Lactobacillus buchneri (G199A and Q222A, see table 9A for a description of strains M23016 and M23017) were also expressed in Saccharomyces cerevisiae under the control of the tef2 promoter and their resulting NAD+ and NADP+ activity was compared to control yeast strains. The results associated with these mutated enzymes is shown in FIG. 12.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Hou J, Scalcinati G, Oldiges M, Vemuri G N. Metabolic impact of increased NADH availability in Saccharomyces cerevisiae. Appl Environ Microbiol. 2010 February; 76(3):851-9.
Serov A E, Popova A S, Fedorchuk V V, Tishkov V I. Engineering of coenzyme specificity of formate dehydrogenase from Saccharomyces cerevisiae. Biochem J. 2002 Nov. 1; 367(Pt 3):841-7.
WO2012138942
Wu W, Dunming Z, Ling H. Site-saturation mutageneis of formate dehydrogenase from Candida bodinii creasing effective NAPD+-dependent FDH enzymes. J Mol Catal B: Enz 2009 61.3: 157-161.

What is claimed is:

1. A recombinant yeast host cell having (i) a first genetic modification for increasing formate production, when compared to a corresponding native yeast host cell and (ii) a source of formate dehydrogenase activity, wherein the source of formate dehydrogenase activity is:
an internal source of formate dehydrogenase activity provided by a second genetic modification; and/or
an external source of formate dehydrogenase activity provided by a further yeast host cell having a third genetic modification.

2. The recombinant yeast host cell of claim 1, wherein the first genetic modification comprises introducing one or more first heterologous nucleic acid molecule encoding one or more polypeptide having pyruvate formate lyase activity in the recombinant yeast host cell.

3. The recombinant yeast host cell of claim 2, wherein the one or more polypeptide having pyruvate formate lyase activity comprises PFLA, PFLB or a combination thereof.

4. The recombinant yeast host cell of claim 2 or 3, wherein the one or more polypeptide having pyruvate formate lyase activity is from Bifidobacterium sp.

5. The recombinant yeast host cell of claim 4, wherein the one or more polypeptide having pyruvate formate lyase activity is from Bifidobacterium adolescentis.

6. The recombinant yeast host cell of claim 5, wherein the one or more polypeptide having pyruvate formate lyase activity comprises the amino acid sequence of SEQ ID NO: 6, is a variant of the amino acid sequence of SEQ ID NO: 6 having pyruvate formate lyase activity or is a fragment of the amino acid sequence of SEQ ID NO: 6 having pyruvate formate lyase activity.

7. The recombinant yeast host cell of claim 5 or 6, wherein the one or more polypeptide having pyruvate formate lyase activity comprises the amino acid sequence of SEQ ID NO: 7, is a variant of the amino acid sequence of SEQ ID NO: 7 having pyruvate formate lyase activity or is a fragment of the amino acid sequence of SEQ ID NO: 7 having pyruvate formate lyase activity.

8. The recombinant yeast host cell of any one of claims 1 to 7, wherein the second and/or third genetic modification comprises introducing a second or third heterologous nucleic acid molecule encoding a polypeptide having formate dehydrogenase activity.

9. The recombinant host cell of claim 8, wherein the polypeptide having formate dehydrogenase activity is FDH1.

10. The recombinant yeast host cell of claim 8 or 9, wherein the polypeptide having formate dehydrogenase activity uses NAD⁺ as a primary cofactor.

11. The recombinant yeast host cell of claim 10, wherein the polypeptide having formate dehydrogenase activity has the amino acid sequence of SEQ ID NO: 1 or 5, is a variant of the amino acid sequence of SEQ ID NO: 1 or 5 having formate dehydrogenase activity or is a fragment of the amino acid sequence of SEQ ID NO: 1 or 5 having formate dehydrogenase activity.

12. The recombinant yeast host cell of claim 8 or 9, wherein the polypeptide having formate dehydrogenase activity uses NADP⁺ as a primary cofactor.

13. The recombinant yeast host cell of claim 12, wherein the polypeptide having formate dehydrogenase activity has the amino acid sequence of SEQ ID NO: 2, 3, 4, 21, 23, 25, 26 or 27, is a variant of the amino acid sequence of SEQ ID NO: 2, 3, 4, 21, 23, 25, 26 or 27 having formate dehydrogenase activity or is a fragment of the amino acid sequence of SEQ ID NO: 2, 3, 4, 21, 23, 25, 26 or 27 having formate dehydrogenase activity.

14. The recombinant yeast host cell of any one of claims 8 to 13, wherein the second and/or third heterologous nucleic acid molecule further comprises a mitochondrial target sequence operatively associated with the nucleic acid sequence encoding the polypeptide having formate dehydrogenase activity.

15. The recombinant yeast host cell of claim 14, wherein the mitochondrial target sequence is from the CYB2 gene.

16. The recombinant yeast host cell of claim 15, wherein the mitochondrial target sequence has the amino acid sequence of SEQ ID NO: 11, is a variant of the amino acid sequence of SEQ ID NO: 11 or is a fragment of the amino acid sequence of SEQ ID NO: 11.

17. The recombinant yeast host cell of any one of claims 8 to 16, wherein the second and/or third heterologous nucleic acid molecule further comprises a promoter operatively associated with the nucleic acid sequence encoding the polypeptide having formate dehydrogenase activity.

18. The recombinant yeast host cell of claim 17, wherein the promoter comprises at least one of tef2p, ssa1p, adh1p, cdc19p, tpi1p, cyc1p, pgk1p, tdh2p, eno2p, hxt3p, qcr8p, tdh1p, tdh3p or hor7p.

19. The recombinant yeast host cell of any one of claims 1 to 18 expressing native FDH gene(s).

20. The recombinant yeast host cell of any one of claims 1 to 19, wherein the further yeast host cell expresses native FDH gene(s).

21. The recombinant yeast host cell of any one of claims 1 to 18 comprising a fourth genetic modification for invactivating of at least one of the native FDH gene(s).

22. The recombinant yeast host cell of any one of claims 1 to 18 and 21, wherein the further yeast host cell comprises a fifth genetic modification for invactivating of at least one of the native FDH gene(s).

23. The recombinant yeast host cell of claim 19 to 22, wherein the native FDH gene(s) comprises FDH1, FDH2 or both.

24. The recombinant yeast host cell of any one of claims 1 to 23 being from the genus Saccharomyces.

25. The recombinant yeast host cell of any one of claims 1 to 24, wherein the further yeast host cell is from the genus Saccharomyces.

26. The recombinant yeast host cell of claim 24 or 25 being from the species Saccharomyces cerevisiae.

27. The recombinant yeast host cell of any one of claims 24 to 26, wherein the further yeast host cell is from the species Saccharomyces cerevisiae.

28. A combination for fermenting a biomass, the combination comprising the recombinant yeast host cell defined in any one of claims 1 to 27 and the further yeast host cell defined in any one of claims 1 to 27.

29. The combination of claim 28, wherein at least one of the recombinant yeast host cell or the further yeast host cell is provided as a cream.

30. A process for converting a biomass into a fermentation product, the process comprises contacting the biomass with the recombinant yeast host cell defined in any one of claims 1 to 27, optionally in combination with the further yeast host cell defined in any one of claims 1 to 27, or the combination of claim 28 or 29 under condition to allow the conversion of at least a part of the biomass into the fermentation product.

31. The process of claim 30, wherein the biomass comprises corn.

32. The process of claim 31, wherein the corn is provided as a mash.

33. The process of any one of claims 30 to 32, wherein the fermentation product is ethanol.

34. The process of any one of claims 30 to 33 being conducted, at least in part, in the presence of a stressor.

35. The process of claim 34, wherein the stressor in lactic acid, formic acid and/or a bacterial contamination.


SEQUENCE LISTING (30 sequences)

SEQ ID NO: 1 - protein, 375 aa, Saccharomyces cerevisiae
SKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYDYQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 2 - protein, 375 aa, Artificial Sequence
  Mutated QRN FDH1
SKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYQRNELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 3 - protein, 375 aa, Artificial Sequence
  Mutated AR FDH1
SKGKVLLVLYEGGKHAEEQEKLLGCIENELGIRNFIEEQGYELVTTIDKDPEPTSTVDRELKDAEIVITTPFFPAYISRNRIAEAPNLKLCVTAGVGSDHVDLEAANERKITVTEVTGSNVVSVAEHVMATILVLIRNYNGGHQQAINGEWDIAGVAKNEYDLEDKIISTVGAGRIGYRVLERLVAFNPKKLLYYARQELPAEAINRLNEASKLFNGRGDIVQRVEKLEDMVAQSDVVTINCPLHKDSRGLFNKKLISHMKDGAYLVNTARGAICVAEDVAEAVKSGKLAGYGGDVWDKQPAPKDHPWRTMDNKDHVGNAMTVHISGTSLDAQKRYAQGVKNILNSYFSKKFDYRPQDIIVQNGSYATRAYGQKK

SEQ ID NO: 4 - protein, 363 aa, Artificial Sequence
  Mutated QRN FDH3
KIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYQRNALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 5 - protein, 363 aa, Candida boidinii
KIVLVLYDAGKHAADEEKLYGCTENKLGIANWLKDQGHELITTSDKEGETSELDKHIPDADIIITTPFHPAYITKERLDKAKNLKLVVVAGVGSDHIDLDYINQTGKKISVLEVTGSNVVSVAEHVVMTMLVLVRNFVPAHEQIINHDWEVAAIAKDAYDIEGKTIATIGAGRIGYRVLERLLPFNPKELLYYDYQALPKEAEEKVGARRVENIEELVAQADIVTVNAPLHAGTKGLINKELLSKFKKGAWLVNTARGAICVAEDVAAALESGQLRGYGGDVWFPQPAPKDHPWRDMRNKYGAGNAMTPHYSGTTLDAQTRYAEGTKNILESFFTGKFDYRPQDIILLNGEYVTKAYGKHDKK

SEQ ID NO: 6 - protein, 292 aa, Bifidobacterium adolescentis
MSEHIFRSTTRHMLRDSKDYVNQTLMGGLSGFESPIGLDRLDRIKALKSGDIGFVHSWDINTSVDGPGTRMTVFMSGCPLRCQYCQNPDTWKMRDGKPVYYEAMVKKIERYADLFKATGGGITFSGGESMMQPAFVSRVFHAAKQMGVHTCLDTSGFLGASYTDDMVDDIDLCLLDVKSGDEETYHKVTGGILQPTIDFGQRLAKAGKKIWVRFVLVPGLTSSEENVENVAKICETFGDALEHIDVLPFHQLGRPKWHMLNIPYPLEDQKGPSAAMKQRVVEQFQSHGFTVY

SEQ ID NO: 7 - protein, 791 aa, Bifidobacterium adolescentis
MAAVDATAVSQEELEAKAWEGFTEGNWQKDIDVRDFIQKNYTPYEGDESFLADATDKTKHLWKYLDDNYLSVERKQRVYDVDTHTPAGIDAFPAGYIDSPEVDNVIVGLQTDVPCKRAMMPNGGWRMVEQAIKEAGKEPDPEIKKIFTKYRKTHNDGVFGVYTKQIKVARHNKILTGLPDAYGRGRIIGDYRRVALYGVNALIKFKQRDKDSIPYRNDFTEPEIEHWIRFREEHDEQIKALKQLINLGNEYGLDLSRPAQTAQEAVQWTYMGYLASVKSQDGAAMSFGRVSTFFDVYFERDLKAGKITETDAQEIIDNLVMKLRIVRFLRTKDYDAIFSGDPYWATWSDAGFGDDGRTMVTKTSFRLLNTLTLEHLGPGPEPNITIFWDPKLPEAYKRFCARISIDTSAIQYESDKEIRSHWGDDAAIACCVSPMRVGKQMQFFAARVNSAKALLYAINGGRDEMTGMQVIDKGVIDPIKPEADGTLDYEKVKANYEKALEWLSETYVMALNIIHYMHDKYAYESIEMALHDKEVYRTLGCGMSGLSIAADSLSACKYAKVYPIYNKDAKTTPGHENEYVEGADDDLIVGYRTEGDFPLYGNDDDRADDIAKWVVSTVMGQVKRLPVYRDAVPTQSILTITSNVEYGKATGAFPSGHKKGTPYAPGANPENGMDSHGMLPSMFSVGKIDYNDALDGISLTNTITPDGLGRDEEERIGNLVGILDAGNGHGLYHANINVLRKEQLEDAVEHPEKYPHLTVRVSGYAVNFVKLTKEQQLDVISRTFHQGAVVD

SEQ ID NO: 8 - protein, 910 aa, Bifidobacterium  adolescentis
MADAKKKEEPTKPTPEEKLAAAEAEVDALVKKGLKALDEFEKLDQKQVDHIVAKASVAALNKHLVLAKMAVEETHRGLVEDKATKNIFACEHVTNYLAGQKTVGIIREDDVLGIDEIAEPVGVVAGVTPVTNPTSTAIFKSLIALKTRCPIIFGFHPGAQNCSVAAAKIVRDAAIAAGAPENCIQWIEHPSIEATGALMKHDGVATILATGGPGMVKAAYSSGKPALGVGAGNAPAYIDKNVDVVRAANDLILSKHFDYGMICATEQAIIADKDIYAPLVKELKRRKAYFVNADEKAKLEQYMFGCTAYSGQTPKLNSVVPGKSPQYIAKAAGFEILEDATILAAECKEVGENEPLTMEKLAPVQAVLKSDNKEQAFEMCEAMLKHGAGHTAAIHTNDRDLVREYGQRMHACRIIWNSPSSLGGVGDIYNAIAPSLTLGCGSYGGNSVSGNVQAVNLINIKRIARRNNNMQWFKIPAKTYFEPNAIKYLRDMYGIEKAVIVCDKVMEQLGIVDKIIDQLRARSNRVTFRIIDYVEPEPSVETVERGAAMMREEFEPDTIIAVGGGSPMDASKIMWLLYEHPEISFSDVREKFFDIRKRAFKIPPLGKKAKLVCIPTSSGTGSEVTPFAVITDHKTGYKYPITDYALTPSVAIVDPVLARTQPRKLASDAGFDALTHAFEAYVSVYANDFTDGMALHAAKLVWDNLAESVNGEPGEEKTRAQEKMHNAATMAGMAFGSAFLGMCHGMAHTIGALCHVAHGRTNSILLPYVIRYNGSVPEEPTSWPKYNKYIAPERYQEIAKNLGVNPGKTPEEGVENLAKAVEDYRDNKLGMNKSFQECGVDEDYYWSIIDQIGMRAYEDQCAPANPRIPQIEDMKDIAIAAYYGVSQAEGHKLRVQRQGEAATEEASERA

SEQ ID NO: 9 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 10 - protein, 569 aa, Saccharomyces cerevisiae
MKDLKLSNFKGKFISRTSHWGLTGKKLRYFITIASMTGFSLFGYDQGLMASLITGKQFNYEFPATKENGDHDRHATVVQGATTSCYELGCFAGSLFVMFCGERIGRKPLILMGSVITIIGAVISTCAFRGYWALGQFIIGRVVTGVGTGLNTSTIPVWQSEMSKAENRGLLVNLEGSTIAFGTMIAYWIDFGLSYTNSSVQWRFPVSMQIVFALFLLAFMIKLPESPRWLISQSRTEEARYLVGTLDDADPNDEEVITEVAMLHDAVNRTKHEKHSLSSLFSRGRSQNLQRALIAASTQFFQQFTGCNAAIYYSTVLFNKTIKLDYRLSMIIGGVFATIYALSTIGSFFLIEKLGRRKLFLLGATGQAVSFTITFACLVKENKENARGAAVGLFLFITFFGLSLLSLPWIYPPEIASMKVRASTNAFSTCTNWLCNFAVVMFTPIFIGQSGWGCYLFFAVMNYLYIPVIFFFYPETAGRSLEEIDIIFAKAYEDGTQPWRVANHLPKLSLQEVEDHANALGSYDDEMEKEDFGEDRVEDTYNQINGDNSSSSSNIKNEDTVNDKANFEG

SEQ ID NO: 11 - protein, 39 aa, Artificial Sequence
  CYB2 mitochondrial target sequence Saccharomyces cerevisiae
MLKYKPLLKISKNCEAAILRASKTRLNTIRAYGSTVPKS

SEQ ID NO: 12 - DNA, 2733 bp, Bifidobacterium adolescentis
atggcagacgcaaagaagaaggaagagccgaccaagccgactccggaagagaagctcgccgcagccgaggctgaggtcgacgctctggtcaagaagggcctgaaggctcttgatgaattcgagaagctcgatcagaagcaggttgaccacatcgtggccaaggcttccgtcgcagccctgaacaagcacttggtgctcgccaagatggccgtcgaggagacccaccgtggtctggtcgaagacaaggccaccaagaacatcttcgcctgcgagcatgtcaccaactacctggctggtcagaagaccgtcggcatcatccgcgaggacgacgtgctgggcatcgacgaaatcgccgagccggttggcgtcgtcgctggcgtgaccccggtcaccaacccgacctccaccgccatcttcaagtcgctgatcgcactgaagacccgctgcccgatcatcttcggcttccacccgggcgcacagaactgctccgtcgcggccgccaagatcgttcgcgatgccgctatcgcagcaggcgctcctgagaactgtattcagtggatcgagcatccgtccatcgaggccactggcgccctgatgaagcatgatggtgtcgccaccatcctcgccaccggtggtccgggcatggtcaaggccgcatactcctccggcaagccggccctgggcgtcggcgcgggcaatgctccggcatacgttgacaagaacgtcgacgtcgtgcgtgcagccaacgatctgattctttccaagcacttcgattacggcatgatctgcgctaccgagcaggccatcatcgccgacaaggacatctacgctccgctcgttaaggaactcaagcgtcgcaaggcctatttcgtgaacgctgacgagaaggccaagctcgagcagtacatgttcggctgcaccgcttactccggacagaccccgaagctcaactccgtggtgccgggcaagtccccgcagtacatcgccaaggccgccggcttcgagattccggaagacgccaccatccttgccgctgagtgcaaggaagtcggcgagaacgagccgctgaccatggagaagcttgctccggtccaggccgtgctgaagtccgacaacaaggaacaggccttcgagatgtgcgaagccatgctgaagcatggcgccggccacaccgccgccatccacaccaacgaccgtgacctggtccgcgagtacggccagcgcatgcacgcctgccgtatcatctggaactccccgagctccctcggcggcgtgggcgacatctacaacgccatcgctccgtccctgaccctgggctgcggctcctacggcggcaactccgtgtccggcaacgtccaggcagtcaacctcatcaacatcaagcgcatcgctcggaggaacaacaacatgcagtggttcaagattccggccaagacctacttcgagccgaacgccatcaagtacctgcgcgacatgtacggcatcgaaaaggccgtcatcgtgtgcgataaggtcatggagcagctcggcatcgttgacaagatcatcgatcagctgcgtgcacgttccaaccgcgtgaccttccgtatcatcgattatgtcgagccggagccgagcgtggagaccgtcgaacgtggcgccgccatgatgcgcgaggagttcgagccggataccatcatcgccgtcggcggtggttccccgatggatgcgtccaagattatgtggctgctgtacgagcacccggaaatctccttctccgatgtgcgtgagaagttcttcgatatccgtaagcgcgcgttcaagattccgccgctgggcaagaaggccaagctggtctgcattccgacttcttccggcaccggttccgaagtcacgccgttcgctgtgattaccgaccacaagaccggctataagtacccgatcaccgattacgcgctgaccccgtccgtcgctatcgtcgatccggtgctggcacgtactcagccgcgcaagctggcttccgatgctggtttcgatgctctgacccacgcttttgaggcttatgtgtccgtgtatgccaacgacttcaccgatggtatggcattgcacgctgccaagctggtttgggacaacctcgctgagtccgtcaatggcgagccgggtgaggagaagacccgtgcccaggagaagatgcataatgccgccaccatggccggcatggctttcggctccgccttcctcggcatgtgccacggcatggcccacaccattggtgcactgtgccacgttgcccacggtcgtaccaactccatcctcctgccgtacgtgatccgttacaacggttccgtcccggaggagccgaccagctggccgaagtacaacaagtacatcgctccggaacgctaccaggagatcgccaagaaccttggcgtgaacccgggcaagactccggaagagggcgtcgagaacctggccaaggctgttgaggattaccgtgacaacaagctcggtatgaacaagagcttccaggagtgcggtgtggatgaggactactattggtccatcatcgaccagatcggcatgcgcgcctacgaagaccagtgcgcaccggcgaacccgcgtatcccgcagatcgaggatatgaaggatatcgccattgccgcctactacggcgtcagccaggcggaaggccacaagctgcgcgtccagcgtcagggcgaagccgctacggaggaagcttccgagcgcgcctga

SEQ ID NO: 13 - DNA, 2733 bp, Artificial Sequence
  Codon optimized version of SEQ ID NO 12
atggccgacgccaagaagaaagaagaacctactaagccaaccccagaagaaaaattggctgctgctgaagctgaagttgatgctttggttaagaaaggtttgaaggccttggacgaattcgaaaaattggatcaaaagcaagtcgatcacatcgttgctaaagcttcagttgctgctttgaacaaacatttggttttggctaagatggccgttgaagaaactcatagaggtttggttgaagataaggccaccaagaatattttcgcttgtgaacatgtcaccaactatttggctggtcaaaagaccgttggtatcattagagaagatgatgttttgggtatcgacgaaattgctgaaccagttggtgttgttgctggtgttactccagttactaatccaacttctaccgctattttcaagtccttgattgccttgaaaaccagatgcccaattatctttggttttcatccaggtgctcaaaactgttctgttgctgctgctaaaatcgttagagatgctgctattgctgctggtgctccagaaaactgtattcaatggattgaacacccatccattgaagctactggtgctttgatgaagcacgatggtgttgctactattttggctactggtggtccaggtatggttaaggctgcttattcttctggtaaaccagctttgggtgttggtgctggtaatgctccagcttatgttgataagaacgttgatgttgttagagctgccaacgatttgattttgtctaagcacttcgactacggtatgatttgtgctactgaacaagctattatcgccgataaggatatctatgctccattggtcaaagaattgaagagaagaaaggcctacttcgttaatgctgacgaaaaagctaagttggaacagtatatgttcggttgtaccgcttactctggtcaaactccaaagttgaattctgttgttccaggtaagtccccacagtatattgctaaagctgccggtttcgaaattccagaagatgctacaattttggccgctgaatgtaaagaagtcggagaaaacgaaccattgaccatggaaaaattggcaccagttcaagctgttttgaagtccgataacaaagaacaagccttcgaaatgtgcgaagccatgttgaaacatggtgctggtcatactgctgctattcatacaaacgatagagacttggtcagagaatacggtcaaagaatgcatgcctgcagaattatttggaactctccatcttctttgggtggtgttggtgatatctacaatgctattgctccatctttgactttgggttgtggttcttatggtggtaattctgtttccggtaatgttcaagccgtcaacttgattaacatcaagagaatcgctagaagaaacaacaacatgcaatggttcaagattccagctaagacttactttgaacctaacgccatcaagtacctaagagatatgtacggtatcgaaaaggctgttatcgtttgcgataaggtcatggaacaattgggtatcgttgataagatcatcgatcaattgagagccagatctaacagagttaccttcagaatcatcgattacgttgaaccagaaccatctgttgaaacagttgaaaggggtgctgctatgatgagagaagaatttgaacctgataccattattgctgttggtggtggttctccaatggatgcttctaagattatgtggttgttgtacgaacacccagaaatttcattctccgatgtcagagaaaagttcttcgacattagaaagagagcctttaagattccaccattgggtaaaaaggccaagttggtatgtattccaacctcttcaggtactggttctgaagttactccattcgctgttattaccgatcataagactggttacaagtacccaattaccgattatgctttgactccatctgttgctatcgttgatccagttttggctagaactcaacctagaaaattggcttctgatgctggttttgatgctttgacacatgcttttgaagcctacgtttctgtttacgctaacgatttcactgatggtatggctttacatgctgctaaattggtttgggataacttggctgaatccgttaatggtgaaccaggtgaagaaaaaactagagcccaagaaaagatgcataacgctgctactatggctggtatggcatttggttctgcttttttgggtatgtgtcatggtatggctcatacaattggtgctttgtgtcatgttgctcatggtagaactaactccattttgttgccatacgtcatcagatacaacggttctgttcctgaagaacctacatcttggccaaagtacaacaagtatattgccccagaaagataccaagaaatcgctaagaacttgggtgttaatccaggtaaaactcctgaagaaggtgttgaaaatttggctaaggctgtcgaagattacagagataacaagttgggtatgaacaagtccttccaagaatgtggtgttgacgaagattactactggtccattatcgatcaaattggtatgagagcctacgaagatcaatgtgctccagctaatccaagaattccacaaatcgaagatatgaaggatattgctattgccgcttactacggtgtttctcaagctgaaggtcataagttgagagttcaaagacaaggtgaagctgctacagaagaagcttctgaaagagcttaa

SEQ ID NO: 14 - DNA, 879 bp, Bifidobacterium adolescentis
atgtctgaacatattttccgttccacgaccagacacatgctgagggattccaaggactacgtcaatcagacgctgatgggaggcctgtccggattcgaatcgccaatcggcttggaccgtctcgaccgcatcaaggcgttgaaaagcggcgatatcggtttcgtgcactcgtgggacatcaacacttccgtggatggtcctggcaccagaatgaccgtgttcatgagcggatgccctctgcgctgccagtactgccagaatccggatacttggaagatgcgcgacggcaagcccgtctactacgaagccatggtcaagaaaatcgagcggtatgccgatttattcaaggccaccggcggcggcatcactttctccggcggcgaatccatgatgcagccggctttcgtgtcacgcgtgttccatgccgccaagcagatgggagtgcatacctgcctcgacacgtccggattcctcggggcgagctacaccgatgacatggtggatgacatcgacctgtgcctgcttgacgtcaaatccggcgatgaggagacctaccataaggtgaccggcggcatcctgcagccgaccatcgacttcggacagcgtctggccaaggcaggcaagaagatctgggtgcgtttcgtgctcgtgccgggcctcacatcctccgaagaaaacgtcgagaacgtggcgaagatctgcgagaccttcggcgacgcgttggaacatatcgacgtattgcccttccaccagcttggccgtccgaagtggcacatgctgaacatcccatacccgttggaggaccagaaaggcccgtccgcggcaatgaaacaacgtgtggtcgagcagttccagtcgcacggcttcaccgtgtactaa

SEQ ID NO: 15 - DNA, 879 bp, Artificial Sequence
  Codon optimized version of SEQ ID NO 14
atgtccgaacacatcttcagatccactactagacacatgttgagagattccaaggactacgttaatcaaactttgatgggtggtttgtctggtttcgaatctccaattggtttggatagattggacagaatcaaggctttgaagtctggtgatatcggttttgttcattcctgggatattaacacctctgttgatggtccaggtactagaatgactgtttttatgtctggttgcccattgagatgtcaatactgtcaaaatccagacacctggaaaatgagagatggtaaaccagtttactacgaagccatggtcaaaaagattgaaagatacgccgatttgttcaaagctactggtggtggtattactttttctggtggtgaatctatgatgcaaccagcttttgtttccagagtttttcatgctgctaagcaaatgggtgttcatacttgtttggatacctctggttttttgggtgcttcttacactgatgatatggttgatgatatcgacttgtgcttgttggatgttaagtcaggtgatgaagaaacctaccataaggttaccggtggtattttacaacctaccattgatttcggtcaaagattggctaaagccggtaaaaagatctgggttagattcgttttggtcccaggtttgacttcttctgaagaaaatgttgaaaacgtcgccaagatttgtgaaacttttggtgatgccttggaacacattgatgttttgccatttcaccaattgggtagaccaaaatggcacatgttgaatattccatacccattggaagatcaaaagggtccatctgctgctatgaagcaaagagttgttgaacaattccaatcccatggtttcaccgtttactaa

SEQ ID NO: 16 - DNA, 2376 bp, Bifidobacterium adolescentis
atggcagcagttgatgcaacggcggtctcccaggaggaacttgaggctaaggcttgggaaggcttcaccgagggcaactggcagaaggacattgatgtccgcgacttcatccagaagaactacacgccatatgagggcgacgagtccttcctggctgacgccaccgacaagaccaagcacctgtggaagtatctggacgacaactatctgtccgtggagcgcaagcagcgcgtctacgacgtggacacccacaccccggcgggcatcgacgccttcccggccggctacatcgattccccggaagtcgacaatgtgattgtcggtctgcagaccgatgtgccgtgcaagcgcgccatgatgccgaacggcggctggcgtatggtcgagcaggccatcaaggaagccggcaaggagcccgatccggagatcaagaagatcttcaccaagtaccgcaagacccacaacgacggcgtcttcggcgtctacaccaagcagatcaaggtagctcgccacaacaagatcctcaccggcctgccggatgcctacggccgtggccgcatcatcggcgattaccgtcgtgtggccctgtacggcgtgaacgcgctgatcaagttcaagcagcgcgacaaggactccatcccgtaccgcaacgacttcaccgagccggagatcgagcactggatccgcttccgtgaggagcatgacgagcagatcaaggccctgaagcagctgatcaacctcggcaacgagtacggcctcgacctgtcccgcccggcacagaccgcacaggaagccgtgcagtggacctacatgggctacctcgcctccgtcaagagccaggacggcgccgccatgtccttcggccgtgtctccaccttcttcgacgtctacttcgagcgcgacctgaaggccggcaagatcaccgagaccgacgcacaggagatcatcgataacctggtcatgaagctgcgcatcgtgcgcttcctgcgcaccaaggattacgacgcgatcttctccggcgatccgtactgggcgacttggtccgacgccggcttcggcgacgacggccgtaccatggtcaccaagacctcgttccgtctgctcaacaccctgaccctcgagcacctcggacctggcccggagccgaacatcaccatcttctgggatccgaagctgccggaagcctacaagcgcttctgcgcccgaatctccatcgacacctcggccatccagtacgagtccgataaggaaatccgctcccactggggcgacgacgccgccatcgcatgctgcgtctccccgatgcgcgtgggcaagcagatgcagttcttcgccgcccgtgtgaactccgccaaggccctgctgtacgccatcaacggcggacgcgacgagatgaccggcatgcaggtcatcgacaagggcgtcatcgacccgatcaagccggaagccgatggcacgctggattacgagaaggtcaaggccaactacgagaaggccctcgaatggctgtccgagacctatgtgatggctctgaacatcatccattacatgcatgataagtacgcttacgagtccatcgagatggctctgcacgacaaggaagtgtaccgcaccctcggctgcggcatgtccggcctgtcgatcgcggccgactccctgtccgcatgcaagtacgccaaggtctacccgatctacaacaaggacgccaagaccacgccgggccacgagaacgagtacgtcgaaggcgccgatgacgatctgatcgtcggctaccgcaccgaaggcgacttcccgctgtacggcaacgatgatgaccgtgccgacgacatcgccaagtgggtcgtctccaccgtcatgggccaggtcaagcgtctgccggtgtaccgcgacgccgtcccgacccagtccatcctgaccatcacctccaatgtggaatacggcaaggccaccggcgccttcccgtccggccacaagaagggcaccccgtacgctccgggcgccaacccggagaacggcatggactcccacggcatgctgccgtccatgttctccgtcggcaagatcgactacaacgacgctcttgacggcatctcgctgaccaacaccatcacccctgatggtctgggccgcgacgaggaagagcgtatcggcaacctcgttggcatcctggacgccggcaacggccacggcctgtaccacgccaacatcaacgtgctgcgcaaggagcagctcgaggatgccgtcgagcatccggagaagtacccgcacctgaccgtgcgcgtctccggctacgcggtgaacttcgtcaagctcaccaaggaacagcagctcgacgtgatctcccgtacgttccaccagggcgctgtcgtcgactga

SEQ ID NO: 17 - DNA, 2376 bp, Artificial Sequence
  Codon optimized version of SEQ ID NO 16
atggctgctgttgatgctaccgctgtttctcaagaagaattggaagctaaagcttgggaaggttttactgaaggtaactggcaaaaggatatcgatgttagagacttcatccaaaagaactacactccatacgaaggtgatgaatcttttttggctgatgctaccgataagaccaaacatttgtggaaatacttggacgacaactacttgtccgtcgaaagaaaacaaagagtttacgacgttgatactcatactccagctggtattgatgcttttccagctggttatattgattccccagaagttgataacgtcatcgttggtttacaaaccgatgttccatgtaagagggctatgatgccaaatggtggttggagaatggttgaacaagctatcaaagaagccggtaaagaaccagatccagaaatcaagaagatcttcaccaagtacagaaagacccataacgatggtgtttttggtgtttacaccaagcaaatcaaggttgctagacacaacaagattttgactggtttgccagatgcttatggtagaggtagaattatcggtgattatagaagagttgccttgtacggtgttaacgctttgattaagttcaagcaaagagacaaggactccattccatacagaaacgatttcaccgaaccagaaatcgaacattggatcagattcagagaagaacacgacgaacaaatcaaggctttgaagcaattgatcaacttgggtaacgaatacggtttggatttgtctagaccagctcaaactgctcaagaagctgttcaatggacttatatgggttatttggcttccgttaagtctcaagatggtgctgctatgtcttttggtagagtttctaccttcttcgacgtctacttcgaaagagatttgaaggctggtaagattactgaaaccgatgcccaagaaatcatcgataacttggtcatgaagttgagaatcgtcagattcttgagaactaaggattacgatgccattttctctggtgatccatattgggctacttggtctgatgctggttttggtgatgatggtagaactatggttaccaagacctccttcagattattgaacactttgaccttggaacatttgggtccaggtccagaacctaacattactattttttgggacccaaagttgccagaagcttacaaaagattctgcgccagaatttctattgatacctccgctattcaatacgaatccgacaaagaaatcagatctcattggggtgatgatgctgctattgcttgttgtgtttctccaatgagagtcggtaagcaaatgcaatttttcgctgctagagtcaactctgctaaggctttgttgtacgctattaacggtggtagagacgaaatgactggtatgcaagtcatcgataagggtgttatcgatccaatcaaacctgaagctgatggtactttggactacgaaaaggttaaggctaattacgaaaaggccttggaatggttgtctgaaacttatgttatggccttgaacatcatccattacatgcatgataagtacgcctacgaatctattgaaatggccttgcatgacaaagaagtctatagaactttgggttgtggtatgtctggtttgtctattgctgctgattctttgtctgcttgtaagtacgctaaggtttacccaatctacaacaaggatgctaaaactactccaggtcacgaaaacgaatatgttgaaggtgctgatgatgatttgatcgttggttatagaaccgaaggtgactttccattatacggtaacgatgatgatagagctgatgatattgccaagtgggttgtttctactgttatgggtcaagttaagagattgccagtttacagagatgctgttccaacccaatccattttgactattacctccaacgtcgaatacggtaaagctactggtgcttttccatcaggtcataagaaaggtactccatatgctccaggtgctaatccagaaaatggtatggattctcatggtatgttgccatctatgttctccgttggtaagatcgattacaacgatgctttggatggtatttctttgaccaacactattaccccagatggtttgggtagagacgaagaagaaagaatcggtaacttggttggtattttggatgctggtaatggtcatggtctataccatgctaacatcaacgtcttgagaaaagaacaattggaagatgccgttgaacacccagaaaagtatccacatttgaccgttagagtttctggttacgctgttaacttcgtcaagttgaccaaagaacaacaattggatgtcatctccagaacttttcatcaaggtgctgttgttgattaa

SEQ ID NO: 18 - DNA, 1548 bp, Saccharomycopsis fibuligera
atgatcagattgacagtctttttgacagcagtttttgctgcagttgctagttgcgtcccggtggaattggacaaaagaaacactggacatttccaagcttattctggatacacagttgccagatcaaatttcactcaatggattcatgagcaaccagctgtttcttggtattatcttttgcaaaacattgattatccagaaggacaatttaaatctgcaaagccaggcgtggtagttgcttctccatccacctcagaacctgactatttttatcaatggaccagagacactgccattacatttctttcgttgattgccgaggttgaagaccatagctttagcaataccacccttgccaaggtcgtggaatactacatcagcaacacctacactttgcaaagagtttcaaacccaagtggaaatttcgacagtcctaaccacgacggtttgggagaaccaaagttcaatgttgacgacaccgcctacacagcttcttggggcagacctcaaaatgatggcccagctttaagagcttatgccatttccagatatttgaatgctgtggccaaacataacaatggcaaattgttgctcgccggccaaaacggaatcccttattctagtgcttctgacatttattggaaaattattaaaccagacttgcaacatgtcagcacccattggagcacctctggctttgatctttgggaagaaaatcaaggaactcatttcttcactgctttggttcaactcaaagctcttagctacggtattcctttgagtaagacttacaacgaccctggctttacttcctggcttgaaaaacaaaaagatgccttgaactcatacatcaactcctctggattcgtcaactcgggtaaaaaacatattgttgaaagcccacaactttcttctagaggcggtttggacagtgccacctacattgctgccttgatcacccatgacattggtgatgatgacacttacactcctttcaacgtggataattcctatgtgctcaattccctatactacttgttggttgacaacaaaaacagatacaagatcaatggcaactacaaagcaggtgctgcggttggaagatatccagaagacgtctacaatggcgttggaactagcgaaggtaacccatggcaattggctactgcctacgctggtcaaactttctacactttggcttacaactctttgaaaaataaaaagaacttggttatagaaaaactcaattacgacctttacaactcctttattgctgacttgtccaagattgactcttcttatgcttccaaagatagtttgactttgacttatggcagcgacaactataaaaatgttatcaaaagtttgctacaatttggtgactctttcttgaaagttctccttgaccatattgatgacaatggccaactcaccgaggaaatcaacagatacactggtttccaagccggcgctgtctccttgacttggagtagtggcagtttgcttagtgcaaacagagctagaaacaaattgattgaacttctttga

SEQ ID NO: 19 - DNA, 1548 bp, Artificial Sequence
  Codon-optimized version of SEQ ID NO 18
atgatcagattgaccgttttcttgaccgctgtttttgctgctgttgcttcttgtgttccagttgaattggataagagaaacaccggtcatttccaagcttattctggttataccgttaacagatctaacttcacccaatggattcatgaacaaccagctgtttcttggtactacttgttgcaaaacatcgattacccagaaggtcaattcaaatctgctaaaccaggtgttgttgttgcttctccatctacatctgaaccagattacttctaccaatggactagagataccgctattaccttcttgtccttgattgctgaagttgaagatcattctttctccaacactaccttggctaaggttgtcgaatattacatttccaacacctacaccttgcaaagagtttctaatccatccggtaacttcgattctccaaatcatgatggtttgggtgaacctaagttcaacgttgatgatactgcttatacagcttcttggggtagaccacaaaatgatggtccagctttgagagcttacgctatttctagatacttgaacgctgttgctaagcacaacaacggtaaattattattggccggtcaaaacggtattccttattcttctgcttccgatatctactggaagattattaagccagacttgcaacatgtttctactcattggtctacctctggttttgatttgtgggaagaaaatcaaggtactcatttcttcaccgctttggttcaattgaaggctttgtcttacggtattccattgtctaagacctacaatgatccaggtttcacttcttggttggaaaaacaaaaggatgccttgaactcctacattaactcttccggtttcgttaactctggtaaaaagcacatcgttgaatctccacaattgtcatctagaggtggtttggattctgctacttatattgctgccttgatcacccatgatatcggtgatgatgatacttacaccccattcaatgttgataactcctacgttttgaactccttgtattacctattggtcgacaacaagaacagatacaagatcaacggtaactacaaagctggtgctgctgttggtagatatcctgaagatgtttacaacggtgttggtacttctgaaggtaatccatggcaattggctactgcttatgctggtcaaactttttacaccttggcctacaattccttgaagaacaagaagaacttggtcatcgaaaagttgaactacgacttgtacaactccttcattgctgatttgtccaagattgattcttcctacgcttctaaggattctttgactttgacctacggttccgataactacaagaacgttatcaagtccttgttgcaattcggtgactcattcttgaaggttttgttggatcacatcgatgacaacggtcaattgactgaagaaatcaacagatacaccggttttcaagctggtgcagtttctttgacttggtcatctggttctttgttgtctgctaatagagccagaaacaagttgatcgaattattgtga

SEQ ID NO: 20 - DNA, 1710 bp, Saccharomyces cerevisiae
atgaaggatttaaaattatcgaatttcaaaggcaaatttataagcagaaccagtcactggggacttacgggtaagaagttgcggtatttcatcactatcgcatctatgacgggcttctccctgtttggatacgaccaagggttgatggcaagtctaattactggtaaacagttcaactatgaatttccagcaaccaaagaaaatggcgatcatgacagacacgcaactgtagtgcagggcgctacaacctcctgttatgaattaggttgtttcgcaggttctctattcgttatgttctgcggtgaaagaattggtagaaaaccattaatcctgatgggttccgtaataaccatcattggtgccgttatttctacatgcgcatttcgtggttactgggcattaggccagtttatcatcggaagagtcgtcactggtgttggaacagggttgaatacatctactattcccgtttggcaatcagaaatgtcaaaagctgaaaatagagggttgctggtcaatttagaaggttccacaattgcttttggtactatgattgcttattggattgattttgggttgtcttataccaacagttctgttcagtggagattccccgtgtcaatgcaaatcgtttttgctctcttcctgcttgctttcatgattaaactacctgaatcgccacgttggctgatttctcaaagtcgaacagaagaagctcgctacttggtaggaacactagacgacgcggatccaaatgatgaggaagttataacagaagttgctatgcttcacgatgctgttaacaggaccaaacacgagaaacattcactgtcaagtttgttctccagaggcaggtcccaaaatcttcagagggctttgattgcagcttcaacgcaatttttccagcaatttactggttgtaacgctgccatatactactctactgtattattcaacaaaacaattaaattagactatagattatcaatgatcataggtggggtcttcgcaacaatctacgccttatctactattggttcattttttctaattgaaaagctaggtagacgtaagctgtttttattaggtgccacaggtcaagcagtttcattcacaattacatttgcatgcttggtcaaagaaaataaagaaaacgcaagaggtgctgccgtcggcttatttttgttcattacattctttggtttgtctttgctatcattaccatggatatacccaccagaaattgcatcaatgaaagttcgtgcatcaacaaacgctttctccacatgtactaattggttgtgtaactttgcggttgtcatgttcaccccaatatttattggacagtccggttggggttgctacttattttttgctgttatgaattatttatacattccagttatcttctttttctaccctgaaaccgccggaagaagtttggaggaaatcgacatcatctttgctaaagcatacgaggatggcactcaaccatggagagttgctaaccatttgcccaagttatccctacaagaagtcgaagatcatgccaatgcattgggctcttatgacgacgaaatggaaaaagaggactttggtgaagatagagtagaagacacctataaccaaattaacggcgataattcgtctagttcttcaaacatcaaaaatgaagatacagtgaacgataaagcaaattttgagggttga

SEQ ID NO: 21 - protein, 398 aa, Lactobacillus buchneri
MTKVLAVLYPDPVDGFPPKYVRDDIPKITHYPDGSTVPTPEGIDFKPGELLGSVSGGLGLKKYLESKGVEFVVTSDKEGPDSVFEKELPTADVVISQPFWPAYLTADLIDKAKKLKLAITAGIGSDHVDLNAANEHNITVAEVTYSNSVSVAEAEVMQLLALVRNFIPAHDIVKAGGWNIADAVSRAYDLEGMTVGVIGAGRIGRAVLERLKPFGVKLVYNQRHQLPDEVENELGLTYFPDVHEMVKVVDAVVLAAPLHAQTYHLFNDEVLATMKRGAYIVNNSRGEEVDRDAIVRALNSGQIGGYSGDVWYPQPAPKDHPWRTMPNEAMTPHMSGTTLSAQARYAAGAREILEDFLEDKPIRPEYLIAQGGSLAGTGAKSYTVKKGEETPGSGEAEK

SEQ ID NO: 22 - DNA, 1197 bp, Artificial Sequence
  Codon optimized DNA sequence encoding MP1180
atgaccaaagttttggctgtcttgtatccagatccagttgatggttttccacctaagtatgttagagatgacattccaaagatcactcactatccagatggttctactgttccaactccagaaggtattgattttaaaccaggtgagttgttgggttctgtttctggtggtttgggtttgaaaaagtacttggaatctaagggtgttgaattcgttgtcacctctgacaaagaaggtccagattccgtttttgagaaagaattgccaactgccgatgtcgttatttctcaaccattttggccagcttatttgaccgctgatttgattgataaggccaagaaattgaagttggctattactgctggtatcggttctgatcatgttgatttgaatgctgccaacgaacataacattaccgttgctgaagttacctactccaattctgtttcagttgccgaagcagaagtcatgcaattattggctttggtcagaaacttcatcccagctcatgatattgtcaaagctggtggttggaatattgctgatgctgtttctagagcttacgacttggaaggtatgactgttggtgttattggtgctggtagaattggtagagctgttttggaaagattgaagccatttggtgttaagttggtctacaaccagagacatcaattgccagatgaagtcgaaaatgaattgggcttgacttactttccagatgttcacgaaatggttaaggttgttgatgcagttgttttagctgctccattgcatgctcaaacttaccatttgttcaacgatgaagtcttggctactatgaagagaggtgcttacatcgttaacaactctagaggtgaagaggttgatagagatgctatagttagagccttgaactctggtcaaattggtggttattctggtgatgtttggtatccacaaccagctccaaaagatcatccttggagaactatgccaaatgaagctatgactccacatatgtctggtactactttgtctgctcaagctagatatgctgctggtgctagagaaattttggaagatttcttggaggacaagccaatcagaccagaatatttgattgctcaaggtggttctttggctggtactggtgctaaatcttacactgttaagaagggtgaagaaactccaggttctggtgaagctgaaaagtaa

SEQ ID NO: 23 - protein, 391 aa, Granulicella mallensis
MAKVLCVLYDDPTSGYPPLYARNAIPKIERYPDGQTVPNPKHIDFVPGELLGCVSGELGLRSYLEDLGHTFIVTSDKEGPNSVFEKELPDADIVISQPFWPAYLTAERIAKAKKLKLALTAGIGSDHVDLNAAIKAGITVAEETFSNGICVAEHAVMMILALVRNYLPSHKIAEEGGWNIADCVSRSYDLEGMHVGTVAAGRIGLAVLRRLKPFDVKLHYTARHRSPRAIEDELGLTYHATAEEMAEVCDVISIHAPLYPATEHLFNAKVLNKMRHGSYLVNTARAEICDRDDIVRALESGQLAGYAGDVWFPQPAPANHPWRNMPHNGMTPHMSGSSLSGQARYAAGTREILECWFENRPIRDEYLIVSNGKLAGTGAKSYGVGEAPKGK

SEQ ID NO: 24 - DNA, 1176 bp, Artificial Sequence
  Codon optimized DNA sequence encoding MP1179
atggctaaggttttgtgtgtcttgtacgatgatccaacttctggttatccaccattatacgctagaaacgccattccaaagattgaaagatatccagatggtcagactgtcccaaatccaaagcacattgattttgtcccaggtgaattattgggttgcgtttctggtgaattgggtttgagatcttacttggaagatttgggtcataccttcatcgttacctctgacaaagaaggtccaaactccgtctttgaaaaagaattgccagatgccgatatcgtgatttctcaaccattttggccagcttatttgaccgctgaaagaatagctaaagccaagaaattgaagttggctttgactgctggtatcggttctgatcatgttgatttgaatgctgctattaaggccggtattactgttgctgaagaaactttctctaacggtatttgcgttgctgaacatgccgttatgatgattttggctttggtcagaaattacctgccatctcataagatagctgaagaaggtggttggaacattgctgattgtgtttctagatcctacgacttggaaggtatgcatgttggtacagttgctgctggtagaattggtttagctgttttgagaagattgaagccattcgatgttaagttgcattacaccgctagacatagatctccaagagctattgaagatgagttgggtttaacttaccatgctactgcagaagaaatggccgaagtttgtgatgttatttctattcacgctccattatacccagctaccgaacatttgtttaatgccaaggttttgaacaagatgaggcacggttcttatttggttaatactgctagagccgaaatctgcgatagagatgatatagttagagccttggaatctggtcaattggctggttatgctggtgatgtttggtttccacaaccagctccagctaatcatccttggagaaatatgccacataatggtatgactccacacatgtctggttcttcattgtctggtcaagctagatatgctgcaggtactagagaaattttggaatgctggtttgaaaacagaccaatcagggatgaatacctgatcgtttccaatggtaaattagctggtactggtgctaaatcttatggtgttggtgaagctccaaagggcaagtaa

SEQ ID NO: 25 - protein, 398 aa, Lactobacillus buchneri
MTKVLAVLYPDPVDGFPPKYVRDDIPKITHYPDGSTVPTPEGIDFKPGELLGSVSGGLGLKKYLESKGVEFVVTSDKEGPDSVFEKELPTADVVISQPFWPAYLTADLIDKAKKLKLAITAGIGSDHVDLNAANEHNITVAEVTYSNSVSVAEAEVMQLLALVRNFIPAHDIVKAGGWNIADAVSRAYDLEGMTVGVIAAGRIGRAVLERLKPFGVKLVYNQRHQLPDEVENELGLTYFPDVHEMVKVVDAVVLAAPLHAQTYHLFNDEVLATMKRGAYIVNNSRGEEVDRDAIVRALNSGQIGGYSGDVWYPQPAPKDHPWRTMPNEAMTPHMSGTTLSAQARYAAGAREILEDFLEDKPIRPEYLIAQGGSLAGTGAKSYTVKKGEETPGSGEAEK

SEQ ID NO: 26 - protein, 398 aa, Lactobacillus buchneri
MTKVLAVLYPDPVDGFPPKYVRDDIPKITHYPDGSTVPTPEGIDFKPGELLGSVSGGLGLKKYLESKGVEFVVTSDKEGPDSVFEKELPTADVVISQPFWPAYLTADLIDKAKKLKLAITAGIGSDHVDLNAANEHNITVAEVTYSNSVSVAEAEVMQLLALVRNFIPAHDIVKAGGWNIADAVSRAYDLEGMTVGVIGAGRIGRAVLERLKPFGVKLVYNARHQLPDEVENELGLTYFPDVHEMVKVVDAVVLAAPLHAQTYHLFNDEVLATMKRGAYIVNNSRGEEVDRDAIVRALNSGQIGGYSGDVWYPQPAPKDHPWRTMPNEAMTPHMSGTTLSAQARYAAGAREILEDFLEDKPIRPEYLIAQGGSLAGTGAKSYTVKKGEETPGSGEAEK

SEQ ID NO: 27 - protein, 386 aa, Bacillus stabilis
MATVLCVLYPDPVDGYPPHYVRDTIPVITRYADGQTAPTPAGPPGFRPGELVGSVSGALGLRGYLEAHGHTLIVTSDKDGPDSEFERRLPDADVVISQPFWPAYLTAERIARAPKLRLALTAGIGSDHVDLDAAARAHITVAEVTGSNSISVAEHVVMTTLALVRNYLPSHAIAQQGGWNIADCVSRSYDVEGMHFGTVGAGRIGLAVLRRLKPFGLHLHYTQRHRLDAAIEQELGLTYHADPASLAAAVDIVNLQIPLYPSTEHLFDAAMIARMKRGAYLINTARAKLVDRDAVVRAVTSGHLAGYGGDVWFPQPAPADHPWRAMPFNGMTPHISGTSLSAQARYAAGTLEILQCWFDGRPIRNEYLIVDGGTLAGTGAQSYRLT

SEQ ID NO: 28 - DNA, 1158 bp, Artificial Sequence
  Codon-optimized sequence encoding SEQ ID NO 27
atggctaccgttttgtgtgtcttgtatccagatccagttgatggttatccaccacattatgttagagataccattccagttattaccagatacgctgatggtcaaactgctccaactccagctggtccaccaggttttagaccaggtgaattggttggttctgtttctggtgctttgggtttgagaggttatttggaagctcatggtcatactttgatcgttacctctgataaggatggtccagattctgaattcgaaagaagattgccagacgccgatgttgttatttctcaaccattttggccagcttacttgaccgctgaaagaattgctagagcaccaaaattgagattggctttgactgctggtattggttctgatcatgttgatttggatgctgctgctagagcccatattactgttgctgaagttactggttccaactctatttcagttgccgaacacgttgttatgactactttggctttggtcagaaactacttgccatctcatgctattgctcaacaaggtggttggaatattgctgattgtgtctctagatcctacgatgttgaaggtatgcattttggtactgttggtgctggtagaattggtttggctgttttgagaagattgaagccatttggtttacacttgcactacacccaaagacatagattggatgcagctatcgaacaagaattgggtttaacttatcatgctgatccagcttcattggctgctgctgttgatatagttaacttgcaaatcccattatacccatccaccgaacatttgtttgatgctgctatgattgctagaatgaagagaggtgcatacttgattaacaccgctagagctaaattggttgatagagatgctgttgttagagctgttacttctggtcatttggctggttatggtggtgatgtttggtttccacaaccagctccagctgatcatccttggagagctatgccttttaatggtatgactccacatatctccggtacatctttgtctgctcaagctagatatgctgctggtactttggaaatattgcaatgttggtttgacggtagaccaatcagaaacgaatatttgattgtcgacggtggtactttagctggtactggtgctcaatcttacagattaact

SEQ ID NO: 29 - DNA, 1161 bp, Artificial Sequence
  Codon-optimized sequence encoding SEQ ID NO 27
atggctactgttttgtgtgtcttgtatccagatccagttgatggttatccaccacattatgttagagataccattccagttattaccagatacgctgatggtcaaactgctccaactccagctggtccaccaggttttagaccaggtgaattggttggttctgtttctggtgctttgggtttgagaggttatttggaagctcatggtcatactttgatcgttacctctgataaggatggtccagattctgaatttgagagaagattgccagatgccgatgttgttatttctcaaccattttggccagcttacttgaccgctgaaagaattgctagagcaccaaaattgagattggctttgactgctggtattggttctgatcatgttgatttggatgctgctgctagagcccatattactgttgctgaagttactggttccaactctatttcagttgccgaacacgttgttatgactactttggctttggtcagaaactacttgccatctcatgctattgctcaacaaggtggttggaatattgctgattgtgtctctagatcctacgatgttgaaggtatgcattttggtactgttggtgctggtagaattggtttggctgttttaagaagattgaagccattcggtttacacttgcattacacccaaagacatagattggatgccgctattgaacaagaattgggtttaacttatcatgccgatccagcttcattggctgctgctgttgatatagttaacttgcaaatcccactgtacccatctactgaacatttgtttgatgctgccatgatcgctagaatgaagagaggtgcttatttgattaacaccgctagagctaagttggttgatagagatgctgttgttagagctgttacttctggtcatttggctggttatggtggtgatgtttggtttccacaaccagctccagctgatcatccttggagagctatgccttttaatggtatgactccacatatctccggtacatctttgtctgctcaagctagatatgctgctggtactttggaaatattgcaatgttggtttgacggtaggccaatcagaaatgaatacttgattgtcgatggtggtacattggctggtactggtgctcaatcttacagattaacttaa

SEQ ID NO: 30 - DNA, 1161 bp, Artificial Sequence
  Codon-optimized sequence encoding SEQ ID NO 27
atggctactgttttgtgtgtcttgtatccagatccagttgatggttatccaccacattatgttagagataccattccagttattaccagatacgctgatggtcaaactgctccaactccagctggtccaccaggttttagaccaggtgaattggttggttctgtttctggtgctttgggtttgagaggttatttggaagctcatggtcatactttgatcgttacctctgataaggatggtccagattctgaattcgaaagaagattgccagacgccgatgttgttatttctcaaccattttggccagcttacttgaccgctgaaagaattgctagagcaccaaaattgagattggctttgactgctggtattggttctgatcatgttgatttggatgctgctgctagagcccatattactgttgctgaagttactggttccaactctatttcagttgccgaacacgttgttatgactactttggctttggtcagaaactacttgccatctcatgctattgctcaacaaggtggttggaatattgctgattgtgtctctagatcctacgatgttgaaggtatgcattttggtactgttggtgctggtagaattggtttggctgttttgagaagattgaagccatttggtttacacttgcactacacccaaagacatagattggatgcagctatcgaacaagaattgggtttaacttatcatgctgatccagcttcattggctgctgctgttgatatagttaacttgcaaatcccattatacccatccaccgaacatttgtttgatgctgctatgattgctagaatgaagagaggtgcatacttgattaacaccgctagagctaaattggttgatagagatgctgttgttagagctgttacttctggtcatttggctggttatggtggtgatgtttggtttccacaaccagctccagctgatcatccttggagagctatgccttttaatggtatgactccacatatctccggtacatctttgtctgctcaagctagatatgctgctggtactttggaaatattgcaatgttggtttgacggtagaccaatcagaaacgaatatttgattgtcgacggtggtactttagctggtactggtgctcaatcttacagattaacttaa